(19) United States
(12) Patent Application Publication
(10) Pub. No.: US 2018/0051069 A1
(43) Pub. Date: Feb. 22, 2018

(54) COMPOSITIONS AND METHODS FOR TREATING ALLERGIC INFLAMMATION

(71) Applicant: THE GENERAL HOSPITAL CORPORATION, Boston, MA (US)

(72) Inventors: Robert M. ANTHONY, Cambridge, MA (US); Kai-Ting SHADE, Arlington, MA (US)

(73) Assignee: THE GENERAL HOSPITAL CORPORATION, Boston, MA (US)

(21) Appl. No.: 15/554,814

(22) PCT Filed: Mar. 4, 2016
(86) PCT No.: PCT/US16/20905

§ 371 Date: Aug. 31, 2017

Related U.S. Application Data
(60) Provisional application No. 62/129,228, filed on Mar. 6, 2015.

(51) Int. Cl. C07K 16/16; C07K 16/18; C07K 14/47

(52) U.S. Cl. CPC C07K 16/16; A61K 2039/505; C07K 14/473; C07K 16/18

(57) ABSTRACT

Compositions and methods are provided that relate to inhibiting and/or reducing glycosylation of IgE for the treatment of allergic and atopic disorders. These compositions and methods are based, in part, on the discovery that glycosylation of IgE is a requirement for initiation of the allergic cascade and that a single N-linked site in the C?3 domain of mouse and human IgE, occupied by an oligomannose structure, specifically N394 of the C?3 domain of human IgE is required for the conformational integrity of the IgE.

### CROSS-REFERENCE TO RELATED APPLICATIONS

This application claims benefit under 35 U.S.C. §119(e) of U.S. Provisional Application Ser. No. 62/129,228 filed Mar. 6, 2015, the content of which is herein incorporated by reference in its entirety.

### GOVERNMENT SUPPORT

This invention was made with government support under grant no. U19AI095261 awarded by The National Institutes of Health. The government has certain rights in the invention.

### TECHNICAL FIELD

The technical field relates to the treatment of allergic diseases.

### BACKGROUND

Allergic diseases, including asthma, atopic dermatitis, as well as allergies to food, dust mites, insect venom, pollen, and pet dander, affect more that one in five individuals world wide, constituting a major global health issue. Immunoglobulin ε (IgE) molecules recognizing innocuous antigens, or allergens, are primary initiators of these diseases. IgE binds and sensitizes tissue resident mast cells expressing the high affinity IgE receptor, FcεRI. Subsequent allergen exposure crosslinks mast cell-bound IgE, resulting in the release of inflammatory mediators, and initiation of the allergic cascade.

### SUMMARY

The compositions and methods described herein are based, in part, on the discovery that glycosylation of IgE is a requirement for initiation of the allergic cascade. As demonstrated herein, this glycosylation requirement was mapped to a single N-linked site in the Cε3 domain of mouse and human IgE, occupied by an oligomannose structure. This glycan is required for the conformational integrity of the IgE, and its removal ablates interactions with FcεRI, thereby preventing allergic reactions and likely reducing IgE tissue half-life. The findings described herein demonstrate a functional requirement for glycosylation of IgE, supporting a close evolutionary relationship shared by immunoglobulin classes²⁵. Because of its essential role in regulating IgE secondary structure, FcεRI binding, mast cell activation, and consequently triggering of anaphylaxis, the oligomannose glycan on N394 in Ce3 of human IgE provides a novel therapeutic target for atopic and allergic diseases.

Accordingly, provided herein are methods of inhibiting or blocking functional activity of IgE comprising administering to a subject in need thereof an inhibitor of glycosylation or an IgE-specific glycosylation inhibitor.

Also provided herein in some aspects are methods of treating an allergic or atopic disease in a subject in need thereof comprising administering to a subject in need thereof an inhibitor of glycosylation or an IgE-specific glycosylation inhibitor, wherein said inhibitor blocks functional activity of IgE, thereby treating an allergic or atopic disease.

In some embodiments of these methods and all such methods described herein, the IgE-specific glycosylation inhibitor blocks or inhibits glycosylation at the glycosylation sequon at N394 of IgE.

In some embodiments of these methods and all such methods described herein, the IgE-specific glycosylation inhibitor blocks or inhibits the glycosylation sequon at N394 of human IgE and prevents or inhibits IgE-mediated signaling.

In some embodiments of these methods and all such methods described herein, the IgE-specific inhibitor is an antibody or antigen-binding fragment thereof that specifically binds or is directed against the glycosylation sequon at N394 of human IgE.

In some embodiments of these methods and all such methods described herein, the IgE-specific inhibitor is an antibody or antigen-binding fragment thereof that specifically binds or is directed against a site on a target in the proximity of the glycosylation sequon at N394 of IgE, thereby providing steric hindrance for the interaction of IgE with its receptor.

In some embodiments of these methods and all such methods described herein, the antibody or antigen-binding fragment thereof that specifically binds or is directed against the glycosylation sequon at N394 of IgE, does not bind to IgE when bound to either of the Fc receptors.

In some embodiments of these methods and all such methods described herein, the IgE-specific inhibitor is a small molecule compound that binds to the glycosylation sequon at N394 of IgE, thereby inhibiting or preventing IgE binding to either of the Fc receptors.

In some embodiments of these methods and all such methods described herein, the small molecule inhibitor specifically binds the glycosylation sequon at N394 of IgE when glycosylated or specifically binds the glycosylation sequon at N394 of IgE when unglycosylated, and prevents the glycosylation at N394.

In some embodiments of these methods and all such methods described herein, the glycosylation inhibitor is selected from tunicamycin, a tunicaymycin homolog, streptovirudin, mycospocidin, amphomycin, tsushimycin, antibiotic 24010, antibiotic MM 19290, bacitracin, corynetoxin, showdomycin, duimycin, 1-deoxymannonojirimycin, deoxynojirimycin, N-methyl-1-dexoymannojirimycin, brefeldin A, a glucose analog, a mannose analog, 2-deoxy-D-glucose, 2-deoxyglucose, D-(+)-mannose, D-(+) galactose, 2-deoxy-2-fluoro-D-glucose, 1,4-dideoxy-1,4-imino-D-mannitol (DIM), fluoroglucose, fluoromannose, UDP-2-deoxyglucose, GDP-2-deoxyglucose, a hydroxymethylglutaryl-CoA reductase inhibitor, 25-hydroxycholesterol, hydroxycholesterol, swainsonine, cycloheximide, puromycin, actinomycin D, monensin, m-Chlorocarbonyl-cyanide phenylhydrazone (CCCP), compactin, dolichyl-phosphoryl-2-deoxyglucose, N-Acetyl-D-Glucosamine, hypoxanthine, thymidine, cholesterol, glucosamine, mannosamine, castanospermine, glutamine, bromoconduritol, conduritol epoxide, a conduritol derivative, aglycosylmethyl-p-nitrophenyltriazene, β-Hydroxynorvaline, threo-β-fluoroasparagine, D-(+)-Gluconic acid.delta.-lactone, di(2-ethyl hexyl)phosphate, tributyl phosphate, dodecyl phosphate, 2-dimethylamino ethyl ester of (diphenyl methyl)-phosphoric acid, [2-(diphenyl phosphinyloxy)ethyl]trimethyl ammonium iodide, iodoacetate, fluoroacetate, Kinefusine, Australine, Castanospermine, Deoxynojirimycin, Deoxymannojirimycin, Swainsoninne, and Mannostatin A.

In some embodiments of these methods and all such methods described herein, the glycosylation inhibitor is selected from an inhibitor of N-linked glycan biosynthesis, an inhibitor of N-linked glycan N-acetylglucosaminyl transferase, an inhibitor of N-linked glycan fucosyl transferase, an inhibitor of N-linked glycan galactosyl transferase, an inhibitor of N-linked glycan sialyl transferase, an inhibitor of N-linked glycan sulfotransferase, an inhibitor of N-linked glycanglycophosphotransferase, or a combination thereof.

In some embodiments of these methods and all such methods described herein, the glycosylation inhibitor is not EndoS.

In some embodiments of these methods and all such methods described herein, the allergic or atopic disorder is selected from allergic asthma, eczema, allergic rhinitis or coryza, hay fever, bronchial asthma, urticaria (hives), gastrointestinal allergies, eosinophilia, conjunctivitis, and glomerulonephritis.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning. A Laboratory Manual, Cold Spring Harbor Laboratory Press, Cold Spring Harbor, N.Y., USA (1982); Sambrook et al., Molecular Cloning. A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

“Glycosylation,” as used herein, refers to a reaction in which a carbohydrate, i.e. a glycosyl donor, is attached to a hydroxyl or other functional group of another molecule (a glycosyl acceptor).

“N-linked glycosylation,” as used herein, is the attachment of the sugar molecule oligosaccharide known as glycan to a nitrogen atom (amide nitrogen of asparagine (Asn/N) residue of a protein).

A “glycosylation sequon,” as used herein, refers to the consensus sequence at which a sugar molecule is attached in N-linked glycosylation comprising Asn-X-Ser (SEQ ID NO: 1), Asn-X-Thr (SEQ ID NO: 2), or Asn-X-Cys (SEQ ID NO: 3), wherein X is an amino acid other than proline.

“Decreased/decreasing glycosylation,” “reduced/reducing glycosylation” or “inhibited/inhibiting glycosylation” as used interchangeably herein, encompass where a polypeptide or protein comprises at least one more unglycosylated (i.e., aglycosylated) site, that is, a completely unoccupied glycan site with no carbohydrate moiety attached thereto, or comprises at least one carbohydrate moiety less at the same potential glycosylation site than an otherwise identical polypeptide or protein which is produced by a cell under otherwise identical conditions but in the absence of a glycosylation inhibiting substance or compound.

“Glycosylation inhibitor” or “glycosylation-inhibiting compound,” as the terms are used herein, refer to a substance or compound where a polypeptide or protein produced in the presence of the substance or compound comprises either at least one unglycosylated site or comprises at least one carbohydrate moiety less at the same site than an otherwise identical polypeptide or protein which is produced by an otherwise identical cell under otherwise identical conditions but in the absence of the substance or compound. Glycosylation inhibitors include, but are not limited to, tunicamycin, tunicaymycin homologs, streptovirudin, mycospocidin, amphomycin, tsushimycin, antibiotic 24010, antibiotic MM 19290, bacitracin, corynetoxin, showdomycin, duimycin, 1-deoxymannonojirimycin, deoxynojirimycin, N-methyl-1-dexoymannojirimycin, brefeldin A, glucose and mannose analogs, 2-deoxy-D-glucose, 2-deoxyglucose, D-(+)-mannose, D-(+) galactose, 2-deoxy-2-fluoro-D-glucose, 1,4-dideoxy-1,4-imino-D-mannitol (DIM), fluoroglucose, fluoromannose, UDP-2-deoxyglucose, GDP-2-deoxyglucose, hydroxymethylglutaryl-CoA reductase inhibitors, 25-hydroxycholesterol, hydroxycholesterol, swainsonine, cycloheximide, puromycin, actinomycin D, monensin, m-Chlorocarbonyl-cyanide phenylhydrazone (CCCP), compactin, dolichyl-phosphoryl-2-deoxyglucose, N-Acetyl-D-Glucosamine, hypoxanthine, thymidine, cholesterol, glucosamine, mannosamine, castanospermine, glutamine, bromoconduritol, conduritol epoxide and conduritol derivatives, glycosylmethyl-p-nitrophenyltriazenes, β-Hydroxynorvaline, threo-β-fluoroasparagine, D-(+)-Gluconic acid δ-lactone, di(2-ethyl hexyl)phosphate, tributyl phosphate, dodecyl phosphate, 2-dimethylamino ethyl ester of (diphenyl methyl)-phosphoric acid, [2-(diphenyl phosphinyloxy)ethyl]trimethyl ammonium iodide, iodoacetate, and/or fluoroacetate.

As used herein, the terms “deglycosylated” “reduced glycosylation,” and “partially glycosylated” protein denotes a protein, such as an IgE protein, that has one or more sugars removed and/or missing from and/or not added to the glycan structure of a fully glycosylated instance of the protein and in which the protein substantially retains its native conformation/folding. A “deglycosylated” protein includes a partially glycosylated protein in which the deglycosylation process leaves a monoglycosylation, a diglycosylation or a triglycosylation at one or more glycosylation sites present on the glycoprotein, relative to the fully glycosylated instance of the protein.

As used herein, a “partially glycosylated” protein includes a “deglycosylated” protein in which one or more sugars are retained at each glycosylation site, and each partial glycosylation site contains a smaller glycan structure (containing fewer sugar units) as compared to the site in a fully glycosylated instance of the glycoprotein, and the partially glycosylated protein substantially retains its native conformation/folding. A “partially glycosylated” protein is generated by partial deglycosylation of the glycan structure of at least one glycosylation site of a fully glycosylated instance of the glycoprotein.

As used herein, the terms “IgE-specific glycosylation inhibitor” or “inhibitor of IgE glycosylation” refer to a molecule or agent that significantly blocks, inhibits, reduces, or interferes with IgE (such as human IgE) biological or functional activity in vitro, in situ, and/or in vivo, including activity of downstream pathways mediated by IgE binding and signaling, such as, for example, elicitation of a cellular response to IgE, such as anaphylaxis, and disorders or conditions associated with the cellular response to IgE, by reducing, inhibiting, or decreasing IgE glycosylation such that an IgE molecule is partially deglycosylated or has reduced glycosylation, as those terms are defined herein. Exemplary IgE-specific glycosylation inhibitors contemplated for use in the various aspects and embodiments described herein include, but are not limited to, anti-IgE antibodies or antigen-binding fragments thereof that specifically bind to the glycosylation sequon at N394 of IgE and inhibit/reduce/block IgE-mediated signaling; and small molecule agents that target or specifically bind to the glycosylation sequon at N394 of IgE and reduce IgE half-life and/or inhibit/reduce/block IgE-mediated signaling, via, for example, blocking/inhibiting binding of IgE to the Fcε receptors.

As used herein, antibodies or antigen-binding fragments thereof include monoclonal, human, humanized or chimeric antibodies, single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

The terms “antibody fragment” or “antigen-binding fragment” include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

The terms “specificity,” “specifically binds,” or “specific for” refers to the number of different types of antigens or antigenic determinants to which a particular antibody or antigen-binding fragment thereof can bind. Accordingly, an antibody or antigen-binding fragment thereof as defined herein is said to be “specific for” a first target or antigen compared to a second target or antigen when it binds to the first antigen with an affinity, and suitably expressed, for example as a K_{D} value) that is at least 10 times, such as at least 100 times, and preferably at least 1000 times, and up to 10,000 times or more better than the affinity with which said amino acid sequence or polypeptide binds to another target or polypeptide. Preferably, when an antibody or antigen-binding fragment thereof is “specific for” a target or antigen, compared to another target or antigen, it is directed against said target or antigen, but not directed against such other target or antigen.

As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da

As used herein, in regard to glycosylation inhibitors and IgE-specific glycosylation inhibitor agents described herein, “modulating” or “to modulate” generally means reducing or inhibiting the functional activity of IgE, as measured using a suitable in vitro, cellular or in vivo assay, such as those described herein in the Examples, by at least 5%, at least 10%, at least 25%, at least 50%, at least 60%, at least 70%, at least 80%, or 90% or more, compared to activity of the target or antigen in the same assay under the same conditions but without the presence of the inhibitor agents described herein. Specifically, as used herein, “functional activities of IgE” that are modulated using the glycosylation inhibitors and IgE-specific glycosylation inhibitor agents described herein include binding of IgE to FcεRI or FcεRII, IgE half-life, IgE secondary structure, mast cell activation, and/or triggering of anaphylaxis.

### BRIEF DESCRIPTION OF THE FIGURES

FIGS. 1A-1F demonstrate that N-linked glycosylation is essential for IgE-mediated allergic inflammation. FIGS. 1A-1B. Quantification of PCA vascular leakage and representative ear images are shown following treatment with PBS, WT or PNG-polyclonal mIgE specific for OVA or peanut extracts (n=8 per group) (FIG. 1A), or monoclonal mIgE specific for OVA, DNP or TNP (n=8 per group) (FIG. 1B). FIG. 1C. Histograms and mean fluorescent intensity (MFI) of A647-OVA and mFcεRI staining on WT or PNG-αOVA-mIgE primed mBMMCs (n=3). FIG. 1D. β-heximinadase activity following OVA-specific degranulation in LAD2 cell sensitized with WT or PNG-treated αOVA-hIgE (n=3) FIG. 1E. Quantified OVA-induced PCA in mFcεRI^{-/-} or hFcεRI⁺mFcεRI^{-/-} mice sensitized by WT or PNG-αOVA-hIgE (n=8 per group). FIG. 1F. Histograms and MFI of surface A647-OVA and hFcεRI on hFcεRI⁺ HeLa cells sensitized by WT or PNG-αOVA-hIgE to hFcεRI on as assessed by A647-OVA (n=3). Means and standard error of the mean (s.e.m.) are plotted; ****p<0.0001, **p<0.01, *p<0.05; ns, not significant.

FIGS. 2A-2E demonstrate that glycosylation of IgE Cε3 is required for anaphylaxis and FcεRI binding. FIGS. 2A-2B. Schematics of αOVA-mIgE (FIG. 2A) and αOVA-hIgE (FIG. 2B) with N-linked glycosylation consensus sites are shown. Percentages of glycan structures identified by glycopeptide mass spectroscopy at each glycosylation site are shown. Glycans are composed of fucose, N-acetylglucosamine (GlcNAc), mannose, galactose (circles), N-acetylgalactosamine (GalNAc, square) and sialic acid. FIG. 2C. Relative magnitude of OVA-specific PCA vascular leakage following sensitization with PBS, WT- or αOVA-mIgE domain mutants. FIG. 2D. OVA-induced degranulation in LAD2 cells sensitized with WT or aOVA-hIgE glycomutants assayed by I3-heximinadase activity. FIG. 2E. Histograms and MFI of hFcεRI⁺ HeLa cells sensitized with WT, CE1 or CE3 aOVA-hIgE glycosylation mutants and stained with A647-OVA and a-hFcεRI-PE. Mean and s.e.m. of at least two independent experiments are shown; ****p<0.0001, ***p<0.001, *p<0.05; ns, not significant.

FIGS. 3A-3G demonstrate that a single N-linked glycosylation site is indispensable for anaphylaxis and hFcεRI binding. FIG. 3A. OVA-induced PCA vascular leakage in mice sensitized with PBS, WT or aOVA-mIgE CE3 mutants (n=8 per group). FIG. 3B. Scatter plots of CD45⁺c-kit⁺CD11b⁻ mast cells recovered from ears injected with a combination of A488-WT and A568-WT aOVA-mIgE or A488-WT and A568-N384Q aOVA-mIgE the previous day. Percentage of A488 and A568 double positive mast cells are shown (n=4 per group). FIG. 3C. Histograms and MFI of A647-OVA and mFcERI staining on WT or N384Q aOVA-mIgE sensitized mBMMCs assessed by Flow Cytometry (n=3). FIG. 3D. OVA-induced degranulation in LAD2 cells sensitized with WT or aOVA-hIgE glycosylation mutants assayed by I3-heximinadase activity (n=3). FIG. 3E. OVA-induced PCA vascular leakage in the ears of hFcERI⁺/mFcERI^{-/-} mice by PBS, WT or N394Q aOVA-hIgE (n=2-6 per group). FIG. 3F. Binding of WT or N394Q aOVA-hIgE to hFcERI on hFcERI⁺ HeLa cells as assessed by A647-OVA. Representative histograms and MFI of A647-OVA and hFcERI are shown (n=3). FIG. 3G. Binding of increasing concentrations of WT or N394Q aOVA-hIgE to immobilized hFcERIa in vitro. Mean and s.e.m. are plotted; ****p<0.0001, ***p<0.001, **p<0.01, *p<0.05; ns, not significant.

FIGS. 4A-4H demonstrate that removal of the IgE oligomannose glycan abrogates anaphylaxis. FIG. 4A. Quantification of PCA vascular leakage by PBS, WT- or EndoF1-mIgE specific for OVA, DNP or TNP (n=8 per group). FIG. 4B. Scatter plots of dermal mast cells recovered from ears injected with a combination of A488-WT- and A568-WT- or A488-WT- and A568-EndoF1-aOVA-mIgE. Percentage of A488 and A568 double positive mast cells are shown (n=4 per group). FIG. 4C. Histograms and MFI of A647-OVA and mFcERI staining on WT, or EndoF1-mIgE, EndoF1 buffer only-mIgE sensitized mBMMCs as assessed by Flow Cytometry (n=3). d, f3-heximinadase activity following OVA- or strepavidin-stimulation of LAD2 cells sensitized with WT-, EndoF1-, or EndoF1 buffer only aOVA-hIgE or biotinylated hIgE (n=3). FIG. 4E. OVA-induced PCA in mFcERI^{-/-} or hFcεRI/mFcERI^{-/—} mice sensitized with WT-, EndoF1-, or EndoF1 buffer only aOVA-hIgE (n=6 per group). FIG. 4F. Histograms and MFI of A647-OVA, A647-streptavidin, and hFcεRI staining on hFcεRI⁺ HeLa cells sensitized with WT, EndoF1-, or EndoF1 buffer only aOVA-hIgE or biotinylated hIgE (n=3). FIG. 4G. Quantitation of WT or EndoF1-treated aOVA-hIgE bound to immobilized hFcεRIa. FIG. 4H. CD spectra of WT, EndoF1-treated or EndoF1 buffer control hIgE recorded in the far UV range (215-240 nm). Mean and s.e.m. are plotted; ****p<0.0001, *p<0.05; ns, not significant.

FIGS. 5A-5F demonstrate that IgE retains recognition to antigen after PNG treatment. FIG. 5A. Schematics of experimental polyclonal IgE sera generation. Balb/c mice received intraperitoneal injection of 10 μg of OVA or peanut extracts in alum every 7 d for 3 weeks. Sera were collected on days indicated by the arrows and depleted with IgG using protein G beads. Some sera were treated with PNGase F before testing its ability to initiate passive cutaneous anaphylaxis (PCA).

FIG. 5B. OVA- or peanut extracts-specific IgE after PNGase F (PNG) treatment. FIG. 5C-5F. Characterization of mouse monoclonal antibodies specific for OVA (FIG. 5C), DNP (FIG. 5D) or TNP (FIG. 5E) and αOVA-hIgE (FIG. 5F) by immunoblotting, lectin blotting, and antigen-specific ELISA after PNG treatment. Error bars represent mean±s.e.m.

FIG. 6 demonstrates analysis of αOVA-mIgE binding to mFcεRI on BMMCs using Flow Cytometry. Representative plots of gating for c-kit+ BMMCs sensitized overnight with no IgE or αOVA-mIgE. Positive staining of A647-OVA, mFcεRI, and anti-mIgE indicates the binding of αOVA-mIgE to mFcεRI on BMMCs.

FIGS. 7A-7B demonstrate domain-specific αOVA-IgE glycosylation mutants retain recognition to OVA. Immunoblotting and OVA-specific ELISA of glycosylation domain-specific mutants for αOVA-mIgE (FIG. 7A) and αOVA-hIgE (FIG. 7B). Error bars represent mean±s.e.m.

FIGS. 8A-8F demonstrate effects of mutating N384 glycosylation site in αOVA-mIgE on binding to OVA and mFcεRI. FIG. 8A. Characterization of individual glycosylation mutants in cε3 domain of αOVA-mIgE by immunoblotting and OVA-specific ELISA. FIG. 8B. OVA-induced PCA vascular leakage in the ears of mice previously sensitized with WT or T386A αOVA-mIgE. FIG. 8C. Gating strategy of dermal mast cells from digested mouse ear. Live dermal mast cells were defined after doublet exclusion and defined size parameters (by SSC and FSC) and gated as CD45+, c-kit+, CD11b-population. FIG. 8D. Representative plots of A568 staining in dermal mast cells from digested mouse ears injected i.d. previous day with A568-WT or A568-N384Q αOVAmIgE. FIG. 8E. Representative plots of A488 and A568 staining in CD11b+Gr-1+ or CD11c+ cells from digested mouse ears previously sensitized with a combination of A488-WT and A568-WT or A488-WT and A568-N384Q αOVA-mIgE. FIG. 8F. Representative plots of A488 and A568 staining in dermal mast cells from digested ears of mFcεRIα−/− mice injected previously with a combination of A488 and A568 WT or A488 WT and A568 N384Q αOVA-mIgE. Error bars represent mean±s.e.m.

FIGS. 9A-9C demonstrate disruption of N394 glycosylation site in αOVA-hIgE abolishes binding to hFcεRI but not OVA. FIG. 9A. Characterization of individual glycosylation mutants in cε3 domain of αOVA-hIgE by immunoblotting and OVA-specific ELISA. FIG. 9B. OVA-induced degranulation (as assessed by β-hexosaminidase in the supernatant) in LAD2 cells that have been primed with WT or T396A αOVA-hIgE. FIG. 9C. Representative histograms and MFI of αOVA-hIgE mutants binding to hFcεRI as assessed by A647-OVA and hFcεRI staining in hFcεRIα+ HeLa cells previously primed with indicated mutants. Error bars represent mean±s.e.m.

FIGS. 10A-10G demonstrate the effect of EndoF1 treatment on IgE conformation and binding to antigen and mast cells. FIGS. 10A-10C. Characterization of mouse monoclonal antibodies specific for OVA (FIG. 10A), DNP (FIG. 10B) or TNP (FIG. 10C) by immunoblotting, lectin blotting, and antigen-specific ELISA after EndoF1 treatment. FIG. 10D. Representative plots of dermal mast cells from digested mouse ears injected i.d. with a combination of A488 and A568 WT or A488 EndoF1-treated WT and A568 WT αOVA-mIgE the previous day. FIG. 10E. Immuno- and lectin blotting of biotinylated hIgE after EndoF1 treatment. FIG. 10F. Characterization of αOVA-hIgE after EndoF1 treatment by immunoblotting, lectin blotting and OVA-specific ELISA. FIG. 10G. Circular dichroism spectra of WT hIgE or hIgE treated with EndoF1 or EndoF1 buffer only in the far UV range (205-245 nm).

### DETAILED DESCRIPTION

Compositions and methods are provided herein that relate to inhibiting and/or reducing glycosylation of IgE for the treatment of allergic and atopic disorders. These compositions and methods described herein are based, in part, on the discovery that glycosylation of IgE is a requirement for initiation of the allergic cascade. As demonstrated herein, this requirement was mapped to a single N-linked site in the Cε3 domain of mouse and human IgE, occupied by an oligomannose structure, specifically N394 of the Cε3 domain of human IgE. This glycan is required for the conformational integrity of the IgE, and its removal ablates interactions with FcεRI, thereby preventing allergic reactions and reducing IgE tissue half-life. The findings described herein demonstrate a functional requirement for glycosylation of IgE, supporting a close evolutionary relationship shared by immunoglobulin classes²⁵. Because of its essential role in regulating IgE secondary structure, FcεRI binding, mast cell activation, and consequently triggering of anaphylaxis, the oligomannose glycan on N394 in Cε3 of human IgE provides a novel therapeutic target for atopic and allergic diseases.

### IgE Biology

Immunoglobulin E (IgE) is a mammal-specific immunoglobulin (Ig) isotype and exists as monomers consisting of two heavy chains (ε chain) and two light chains, with the ε chain comprising 4 Ig-like constant domains (Cε1-Cε4). IgE's main function is immunity to parasites such as parasitic worms, such as Schistosoma mansoni, Trichinella spiralis, and Fasciola hepatica, and during immune defense against certain protozoan parasites such as Plasmodium falciparum.

IgE also plays an essential role in type I hypersensitivity, which causes various allergic diseases, such as allergic asthma, most types of sinusitis, allergic rhinitis, food allergy, and some types of chronic urticaria and atopic dermatitis. IgE also plays a pivotal role in allergic conditions, such as anaphylactic reactions to certain drugs, bee stings, and antigen preparations used in specific desensitization immunotherapy.

IgE primes the IgE-mediated allergic response by binding to Fcε receptors found on the surface of mast cells and basophils. Fc receptors are also found on eosinophils, monocytes, macrophages and platelets in humans. There are two types of Fcε receptors: FcεRI (type I Fcε receptor), the high-affinity IgE receptor, and the FcεRII (type II Fcε receptor), also known as CD23, the low-affinity IgE receptor FcεRI is expressed on mast cells, basophils, and the antigen-presenting dendritic cells in both mice and humans. Binding of antigens to IgE already bound by the FcεRI on mast cells causes cross-linking of the bound IgE and the aggregation of the underlying FcεRI, leading to the degranulation and the release of mediators from the cells. Basophils, upon the cross-linking of their surface IgE by antigens, release type 2 cytokines like interleukin-4 (IL-4) and interleukin-13 (IL-13) and other inflammatory mediators. The low-affinity receptor (FcεRII) is always expressed on B cells, but its expression can be induced on the surfaces of macrophages, eosinophils, platelets, and some T cells by IL-4.

Demonstrated herein is the novel discovery that glycosylation of a specific residue of IgE, N394 of the Cε3 domain of human IgE, is required for the conformational integrity of the IgE, and its removal ablates interactions with FcεRI, thereby preventing allergic reactions and reducing IgE tissue half-life. As demonstrated herein, glycosylation of the N394 residue of the Cε3 domain of human IgE plays an essential role in regulating IgE secondary structure, FcεRI binding, mast cell activation, and consequently triggering of anaphylaxis, thereby providing a novel therapeutic target for atopic and allergic diseases.

Accordingly, provided herein are compositions and methods comprising glycosylation inhibitors for deglycosylating, partially glycosylating, reducing or inhibiting glycosylation of IgE for the treatment of atopic and allergic diseases.

### Glycosylation and Inhibitors Thereof

Glycosylation, as used herein, refers to a reaction in which a carbohydrate, i.e. a glycosyl donor, is attached to a hydroxyl or other functional group of another molecule (a glycosyl acceptor). Glycosylation is a form of co-translational and post-translational modification. Glycans serve a variety of structural and functional roles in membrane and secreted proteins. The majority of proteins synthesized in the rough ER undergo glycosylation. It is an enzyme-directed site-specific process. Glycosylation is also present in the cytoplasm and nucleus as the O-GlcNAc modification.

In eukaryotes, sugar residues are commonly linked to four different amino acid residues. These amino acid residues are classified as O-linked (serine, threonine, and hydroxylysine) and N-linked (asparagine), and the process of such glycosylation is referred to as “O-linked glycosylation” and “N-linked glycosylation,” respectively. O-linked sugars are synthesized in the Golgi or rough Endoplasmic Reticulum (ER) from nucleotide sugars.

As used herein, “N-linked glycosylation” refers to the attachment of a sugar molecule oligosaccharide, known as “glycan,” to a nitrogen atom (amide nitrogen of asparagine (Asn/N) residue of a protein). N-linked-glycans are found in mammals and comprise a plurality of oligosaccharide chains linked to a core protein via a nitrogen atom of an Asparagine (Asn/N) residue. The Asn residue occurs in a tripeptide sequence, also referred to herein as a “glycosylation sequon,” comprising e.g. Asn-X-Ser (SEQ ID NO: 1), Asn-X-Thr (SEQ ID NO: 2), or Asn-X-Cys (SEQ ID NO: 3), wherein X is an amino acid other than proline. In the lumen of the endoplasmic reticulum, a 14-residue precursor oligosaccharide (glucose₃ mannose₉ N-acetylglucosamine₂, (Glc₃Man₉GlcNAc₂)) is transferred to an asparagine residue found in the consensus sequence N-X-S/T (SEQ ID NO: 16) by the enzyme oligosaccharyltransferase. Next, the precursor glycan is trimmed to a high-mannose structure (Man₈₋₉GlcNAc₂) by exoglycosidases while the protein being synthesized is assembled and transported to the Golgi. This structure can be found, for example, on the surface of the HIV envelope protein, gp120. However, the glycan can be further processed as a protein progresses through the secretory pathway. In the cis-Golgi, the mannose residues are trimmed by α1,2 mannosidase-I, yielding the oligomannose structure Man₅GlcNAc₂. The constant chains of IgE antibodies have these oligomannose structures. In the medial-Golgi, N-acetylglucosamine is added by β1,2 N-acetylglucosaminyltransferase-I forming GlcNAc₁Man₅GlcNAc₂, and α 1,2 mannosidase-II further removes mannose residues forming the hybrid glycan structure GlcNAc₁Man₃GlcNAc₂. Next, the core complex biantennary glycan structure (GlcNAc₃Man₃GlcNAc₂) is generated by the transfer of GlcNAc by β,2 N-acetylglucosaminyltransferase-II. This structure is found in the constant region of IgG antibodies, and can be further modified. The core GlcNAc is available for fucosylation by α1,6-fucosyltransferase. Bisecting N-acetylglucosamine is attached to the core by N-acetylglucosaminyltransferase-III. As a protein progresses along the secretory pathway, the glycan can be further modified in the trans-Golgi by the addition of galactose and sialic acid to the arms by β1,4 galactosyltransferase and α2,6 sialyltransferase, respectively. Accordingly, in some embodiments of the aspects described herein, a IgE-specific glycosylation inhibitor can target any of the enzymes involved in IgE glycosylation, such as oligosaccharyltransferase, exoglycosidases, α1,2 mannosidase-I, β1,2 N-acetylglucosaminyltransferase-I, α1,2 mannosidase-II, β,2 N-acetylglucosaminyltransferase-II, α1,6-fucosyltransferase, N-acetylglucosaminyltransferase-III, β1,4 galactosyltransferase, and α2,6 sialyltransferase.

It is known that addition of N-linked carbohydrate chains is important for stabilization of folding, prevention of degradation in the endoplasmic reticulum, oligomerization, biological activity, and transport of glycoproteins. The addition of N-linked oligosaccharides to specific Asn residues plays an important role in regulating the activity, stability or antigenicity of mature proteins of viruses (Opdenakker G. et al FASEB Journal 7, 1330-1337 1993). Studies have also shown that N-linked glycosylation is required for folding, transport, cell surface expression, secretion of glycoproteins (Helenius, A., Molecular Biology of the Cell 5, 253-265 1994), protection from proteolytic degradation and enhancement of glycoprotein solubility (Doms et al., Virology 193, 545-562 1993).

The biosynthesis of the various types of N-linked oligosaccharide structures involves two series of reactions: 1) the formation of the lipid-linked saccharide precursor, Glc₃Man₉(GlcNAc)2-pyrophosphoryl-dolichol, by the stepwise addition of GlcNAc, mannose and glucose to dolich01-P; and 2) the removal of glucose and mannose by membrane-bound glycosidases and the addition of GlcNAc, galactose, sialic acid, and fucose by Golgi-localized glycosyltransferases to produce different complex oligosaccharide structures.

The first four enzymes that function in the processing of N-linked glycoproteins are glycosidases. GlcNAc-phosphotransferase catalyzes the first step in the synthesis of the mannose 6-phosphate determinant. A proper carbohydrate structure greatly facilitates the efficient phosphorylation by GlcNAc-phosphotransferase. The carbohydrate structure coupled to phosphorylation is necessary for the synthesis of a mannose-6-phosphate signal on the GAA molecule is a high mannose N-glycan.

### Glycosylation Inhibitors and IgE-Specific Glycosylation Inhibitors

Inhibitors of glycosylation are contemplated for use according to the compositions and methods described herein. Such inhibitors are used to inhibit/block the glycosylation sequon at N394 of IgE and/or deglycosylate or reduce glycosylation of an IgE molecule in a subject in need thereof. In particular, such glycosylation inhibitors are used to inhibit or block the glycosylation sequon at N394 of IgE, and/or inhibit or remove glycosylation at N394 of the Cε3 domain of human IgE, thereby inhibiting/reducing downstream functional activity of human IgE.

Accordingly, provided herein, in some aspects, are methods of inhibiting or blocking functional activity of IgE comprising administering to a subject in need thereof an inhibitor of glycosylation or an IgE-specific glycosylation inhibitor.

In some aspects, provided herein are methods of treating an allergic or atopic disease in a subject in need thereof comprising administering to a subject in need thereof an inhibitor of glycosylation or an IgE-specific glycosylation inhibitor, thereby inhibiting or blocking functional activity of IgE.

“Decreased/decreasing glycosylation,” “reduced/reducing glycosylation” or “inhibited/inhibiting glycosylation” as used interchangeably herein, encompass where a polypeptide or protein comprises at least one more unglycosylated (i.e., aglycosylated) site, that is, a completely unoccupied glycan site with no carbohydrate moiety attached thereto, or comprises at least one carbohydrate moiety less at the same potential glycosylation site than an otherwise identical polypeptide or protein which is produced by a cell under otherwise identical conditions but in the absence of a glycosylation inhibiting substance or compound.

As used herein, the terms “IgE-specific glycosylation inhibitor” or “inhibitor of IgE glycosylation” refer to a molecule or agent that significantly blocks, inhibits, reduces, or interferes with IgE (such as human IgE) biological or functional activity in vitro, in situ, and/or in vivo, including activity of downstream pathways mediated by IgE binding and signaling, such as, for example, elicitation of a cellular response to IgE, such as anaphylaxis, and disorders or conditions associated with the cellular response to IgE, by reducing, inhibiting, or decreasing IgE glycosylation such that an IgE molecule is partially deglycosylated or has reduced glycosylation, as those terms are defined herein. Exemplary IgE-specific glycosylation inhibitors contemplated for use in the various aspects and embodiments described herein include, but are not limited to, anti-IgE antibodies or antigen-binding fragments thereof that specifically bind to the glycosylation sequon at N394 of IgE and inhibit/reduce/block IgE-mediated signaling; and small molecule agents that target or specifically bind to the glycosylation sequon at N394 of IgE and reduce IgE half-life and/or inhibit/reduce/block IgE-mediated signaling, via, for example, blocking/inhibiting binding of IgE to the Fce receptors.

In some embodiments of the compositions, methods, and uses described herein, an IgE-specific glycosylation inhibitor blocks or inhibits glycosylation at the glycosylation sequon at N394 of IgE.

In some embodiments of the compositions, methods, and uses described herein, an IgE-specific glycosylation inhibitor blocks or inhibits the glycosylation sequon at N394 of IgE and prevents/inhibits IgE-mediated signaling.

In some embodiments of the compositions, methods, and uses described herein, the binding sites of the IgE-specific inhibitors, such as an antibody or antigen-binding fragment thereof, specifically bind or are directed against the glycosylation sequon at N394 of IgE. In some embodiments of the compositions, methods, and uses described herein, the binding sites of the IgE-specific inhibitors specifically bind or are directed against a site on a target in the proximity of the glycosylation sequon at N394 of IgE, in order to provide steric hindrance for the interaction of IgE with its receptor.

As used herein, and as would be understood by one of ordinary skill in the art, the glycosylation sequon at N394 of IgE is based on numbering the amino acids of a human IgE molecule to be inclusive of the variable domain. If numbering begins at the CH1 domain of human IgE, then the equivalent amino acid residue is N275.

Accordingly, in some embodiments of the compositions, methods, and uses described herein, the binding sites of the IgE-specific inhibitors, such as an antibody or antigen-binding fragment thereof, specifically bind or are directed against the glycosylation sequon at N394 of human IgE, based on numbering the amino acids of a human IgE molecule to be inclusive of the variable domain. In some embodiments of the compositions, methods, and uses described herein, the binding sites of the IgE-specific inhibitors, such as an antibody or antigen-binding fragment thereof, specifically bind or are directed against the glycosylation sequon at N275 of human IgE, based on numbering the amino acids of a human IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N384 of mouse IgE, based on numbering the amino acids of a mouse IgE molecule to be inclusive of the variable domain. In some embodiments of the compositions, methods, and uses described herein, the binding sites of the IgE-specific inhibitors, such as an antibody or antigen-binding fragment thereof, specifically bind or are directed against the glycosylation sequon at N261 of mouse IgE, based on numbering the amino acids of a mouse IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N275 of rhesus macaque IgE, based on numbering the amino acids of a rhesus macaque IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N265 of rat IgE, based on numbering the amino acids of a rat IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N271 of rabbit IgE, based on numbering the amino acids of a rabbit IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N271 of dog IgE, based on numbering the amino acids of a dog IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N272 of cat IgE, based on numbering the amino acids of a cat IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N270 of bovine IgE, based on numbering the amino acids of a bovine IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N271 of pig IgE, based on numbering the amino acids of a pig IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N270 of horse IgE, based on numbering the amino acids of a horse IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N263 of opossum IgE, based on numbering the amino acids of an opossum IgE molecule to begin at the CH1 domain.

In some embodiments of the compositions, methods, and uses described herein, the binding sites of the IgE-specific inhibitors, such as an antibody or antigen-binding fragment thereof, specifically bind or are directed against the glycosylation sequon at N276 of platypus IgE, based on numbering the amino acids of a platypus IgE molecule to begin at the CH1 domain.

As used herein, an IgE-specific inhibitor has the ability to reduce the half-life or functional activity of IgE in a subject by at least 5%, at least 10%, at least 20%, at least 30%, at least 40%, at least 50%, at least 60%, at least 70%, at least 80%, at least 90%, at least 95%, at least 98%, at least 99%, or more, relative to the activity or expression level in the absence of the IgE-specific inhibitor.

In some embodiments of the compositions, methods, and uses described herein, the IgE-specific inhibitor is an antibody or antigen-binding fragment thereof that binds to the glycosylation sequon at N394 of IgE, but does not bind to IgE when the IgE is bound to either of its receptors. In other words, in some embodiments of the compositions, methods, and uses described herein, the IgE-specific inhibitor is an antibody or antigen-binding fragment thereof that binds to an epitope found at or near the glycosylation sequon at N394 of circulating IgE, but not available for binding in the IgE when bound to either of the FCε receptors.

IgE-specific antibodies or antigen-binding fragments thereof that are specific for or that selectively bind the glycosylation sequon at N394 of IgE, suitable for use in the compositions and for practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

Examples of antibody fragments encompassed by the terms antibody fragment or antigen-binding fragment include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein.

With respect to a target or antigen, the term “ligand interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen, i.e., IgE molecule. More generally, a “ligand interaction site” can be any site, epitope, antigenic determinant, part, domain or stretch of amino acid residues on a target or antigen to which a binding site of an IgE-specific inhibitor described herein can bind such that the IgE molecule (and/or any pathway, interaction, signalling, biological mechanism or biological effect in which the IgE molecule is involved) is modulated.

In the context of an antibody or antigenbinding fragment thereof, the term “specificity” or “specific for” refers to the number of different types of antigens or antigenic determinants to which a particular antibody or antigen-binding fragment thereof can bind. The specificity of an antibody or antigen-binding fragment or portion thereof can be determined based on affinity and/or avidity. The affinity, represented by the equilibrium constant for the dissociation (K_{D}) of an antigen with an antigen-binding protein, is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, an antibody or antigen-binding fragment thereof as defined herein is said to be “specific for” a first target or antigen compared to a second target or antigen when it binds to the first antigen with an affinity (as described above, and suitably expressed, for example as a K_{D} value) that is at least 10 times, such as at least 100 times, and preferably at least 1000 times, and up to 10.000 times or more better than the affinity with which said amino acid sequence or polypeptide binds to another target or polypeptide. Preferably, when an antibody or antigen-binding fragment thereof is “specific for” a target or antigen, compared to another target or antigen, it is directed against said target or antigen, but not directed against such other target or antigen.

Avidity is the measure of the strength of binding between an antigen-binding molecule and the pertinent antigen. Avidity is related to both the affinity between an antigenic determinant and its antigen binding site on the antigen-binding molecule, and the number of pertinent binding sites present on the antigen-binding molecule. Typically, antigen-binding proteins will bind to their cognate or specific antigen with a dissociation constant (K_{D} of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction is, the lower is its K_{D}. Preferably, a binding site on an IgE-specific inhibitor antibody or antigen-binding fragment thereof described herein will bind to the glycosylation sequon at N394 with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

The term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each antibody in a monoclonal preparation is directed against the same, single determinant on the antigen. It is to be understood that the term “monoclonal antibody” refers to an antibody that is derived from a single clone, including any eukaryotic, prokaryotic, or phage clone, and not the method by which it is produced. The term “monoclonal antibody” as used herein is not limited to antibodies produced through hybridoma technology, and the modifier “monoclonal” is not to be construed as requiring production of the antibody by any particular method. For example, the monoclonal antibodies to be used in accordance with the invention can be made by the hybridoma method first described by Kohler et al., Nature 256:495 (1975), or later adaptations thereof, or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example.

As used herein, the term “chimeric antibody” refers to an antibody molecule in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Chimeric antibody molecules can include, for example, one or more antigen binding domains from an antibody of a mouse, rat, or other species, with human constant regions. A variety of approaches for making chimeric antibodies have been described and can be used to make chimeric antibodies containing the immunoglobulin variable region which recognizes the desired antigen, e.g., IL-27 or NFIL-3. See, for example, Takeda et al., 1985, Nature 314:452; Cabilly et al., U.S. Pat. No. 4,816,567; Boss et al., Tanaguchi et al., European Patent Publication EP171496; European Patent Publication 0173494, United Kingdom patent GB 2177096B).

Humanized forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments of the compositions, methods, and uses comprising any of the IgE-specific antibodies or antigen-binding fragments thereof described herein, the IgE-specific antibody or antigen-binding fragment is an antibody derivative. For example, but not by way of limitation, antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids.

The IgE-specific antibodies and antigen-binding fragments thereof described herein can be generated by any suitable method known in the art. Monoclonal and polyclonal antibodies against, for example, IgE, are known in the art. To the extent necessary, e.g., to generate antibodies with particular characteristics or epitope specificity, the skilled artisan can generate new monoclonal or polyclonal IgE-specific antibodies as briefly discussed herein or as known in the art.

Polyclonal antibodies can be produced by various procedures well known in the art. For example, IgE molecules or fragments thereof comprising the glycosylation sequon at N394, can be administered to various host animals including, but not limited to, rabbits, mice, rats, etc. to induce the production of sera containing polyclonal antibodies specific for the protein. Polyclonal antibodies are preferably raised in animals by multiple subcutaneous (sc) or intraperitoneal (ip) injections of the relevant antigen and an adjuvant. It can be useful to conjugate the antigen to a protein that is immunogenic in the species to be immunized, e.g., keyhole limpet hemocyanin, serum albumin, bovine thyroglobulin, or soy-bean trypsin inhibitor using a bifunctional or derivatizing agent, for example, maleimidobenzoyl sulfosuccinimide ester (conjugation through cysteine residues), N-hydroxy-succinimide (through lysine residues), glutaraldehyde, succinic anhydride, SOCl₂, or R ¹N═C═NR, where R and R¹ are different alkyl groups. Various other adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Suitable adjuvants are also well known to one of skill in the art.

Monoclonal antibodies can be prepared using a wide variety of techniques known in the art including the use of hybridoma, recombinant, and phage display technologies, or a combination thereof. Various methods for making monoclonal antibodies described herein are available in the art. For example, the monoclonal antibodies can be made using the hybridoma method first described by Kohler et al., Nature, 256:495 (1975), or any later developments thereof, or by recombinant DNA methods (U.S. Pat. No. 4,816,567). For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammer-ling, et al., in: Monoclonal Antibodies and T-Cell Hybridomas 563-681 (Elsevier, N.Y., 1981) (said references incorporated by reference in their entireties). Methods for producing and screening for specific antibodies using hybridoma technology are routine and well known in the art. In another example, antibodies useful in the methods and compositions described herein can also be generated using various phage display methods known in the art, such as isolation from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

Human antibodies can be made by a variety of methods known in the art, including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, the contents of which are herein incorporated by reference in their entireties.

Human antibodies can also be produced using transgenic mice which express human immunoglobulin genes, and upon immunization are capable of producing a full repertoire of human antibodies in the absence of endogenous immunoglobulin production. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, the contents of which are herein incorporated by reference in their entireties. In addition, companies such as Abgenix, Inc. (Freemont, Calif.) and Medarex (Princeton, N.J.) can be engaged to provide human antibodies directed against a selected antigen using technology similar to that described above. See also, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992), the contents of which are herein incorporated by reference in their entireties. Alternatively, phage display technology (McCafferty et al., Nature 348:552-553 (1990)) can be used to produce human antibodies and antibody fragments in vitro, from immunoglobulin variable (V) domain gene repertoires from unimmunized donors. Human antibodies can also be generated by in vitro activated B cells (see U.S. Pat. Nos. 5,567,610 and 5,229,275, the contents of which are herein incorporated by reference in their entireties). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

“An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the human heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

As used herein, a “chimeric antibody” refers to a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al, 1986, Bio-Techniques 4:214; Gillies et al., 1989, J. Immunol. Methods 125:191-202; U.S. Pat. Nos. 5,807,715; 4,816,567; and 4,816,397, the contents of which are herein incorporated by reference in their entireties.

“Humanized antibodies,” as the term is used herein, refer to antibody molecules from a non-human species, where the antibodies that bind the desired antigen, i.e., IL-27 or NFIL-3, have one or more CDRs from the non-human species, and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al, 1994, PNAS 91:969-973), and chain shuffling (U.S. Pat. No. 5,565,332), the contents of which are herein incorporated by reference in their entireties. Accordingly, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), the contents of which are herein incorporated by reference in their entireties, by substituting rodent CDRs or CDR sequences for the corresponding sequences of a human antibody. Accordingly, such “humanized” antibodies are chimeric antibodies (U.S. Pat. No. 4,816,567, the contents of which are herein incorporated by reference in its entirety) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The “Fab” fragment contains a variable and constant domain of the light chain and a variable domain and the first constant domain (C_{H}1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

Various techniques have been developed for the production of antibody or antigen-binding fragments. The antibodies described herein can be fragmented using conventional techniques and the fragments screened for utility in the same manner as described above for the whole antibodies. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). For example, Fab and F(ab′)₂ fragments of the bispecific and multispecific antibodies described herein can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the C_{H}1 domain of the heavy chain. However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040. For some uses, including the in vivo use of antibodies in humans as described herein and in vitro proliferation or cytotoxicity assays, it is preferable to use chimeric, humanized, or human antibodies.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

As used herein “complementary” refers to when two immunoglobulin domains belong to families of structures which form cognate pairs or groups or are derived from such families and retain this feature. For example, a V_{H} domain and a V_{L} domain of a natural antibody are complementary; two V_{H} domains are not complementary, and two V_{L} domains are not complementary. Complementary domains can be found in other members of the immunoglobulin superfamily, such as the V_{α}, and V_{β} (or γ and δ) domains of the T-cell receptor. Domains which are artificial, such as domains based on protein scaffolds which do not bind epitopes unless engineered to do so, are non-complementary. Likewise, two domains based on, for example, an immunoglobulin domain and a fibronectin domain are not complementary.

In some embodiments of the compositions, methods, and uses described herein, the IgE-specific inhibitor is a small molecule compound that binds to the glycosylation sequon at N394 of IgE, thereby inhibiting or preventing IgE binding to either of the Fcε receptors. Such small molecule inhibitors include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da. In some embodiments of the compositions, methods, and uses described herein, a small molecule inhibitor comprises a small molecule that specifically binds the glycosylation sequon at N394 of IgE when glycosylated or that specifically binds the glycosylation sequon at N394 of IgE when unglycosylated, and prevents the glycosylation at N394. Exemplary sites of small molecule binding include, but are not limited to, the portion of IgE that binds to either of the Fcε receptors.

In some embodiments of the compositions, methods, and uses described herein, a glycosylation inhibitor includes, but is not limited to, tunicamycin, a tunicaymycin homolog, streptovirudin, mycospocidin, amphomycin, tsushimycin, antibiotic 24010, antibiotic MM 19290, bacitracin, corynetoxin, showdomycin, duimycin, 1-deoxymannonojirimycin, deoxynojirimycin, N-methyl-1-dexoymannojirimycin, brefeldin A, a glucose analog, a mannose analog, 2-deoxy-D-glucose, 2-deoxyglucose, D-(+)-mannose, D-(+) galactose, 2-deoxy-2-fluoro-D-glucose, 1,4-dideoxy-1,4-imino-D-mannitol (DIM), fluoroglucose, fluoromannose, UDP-2-deoxyglucose, GDP-2-deoxyglucose, a hydroxymethylglutaryl-CoA reductase inhibitor, 25-hydroxycholesterol, hydroxycholesterol, swainsonine, cycloheximide, puromycin, actinomycin D, monensin, m-Chlorocarbonyl-cyanide phenylhydrazone (CCCP), compactin, dolichyl-phosphoryl-2-deoxyglucose, N-Acetyl-D-Glucosamine, hypoxanthine, thymidine, cholesterol, glucosamine, mannosamine, castanospermine, glutamine, bromoconduritol, conduritol epoxide, a conduritol derivative, aglycosylmethyl-p-nitrophenyltriazene, β-Hydroxynorvaline, threo-β-fluoroasparagine, D-(+)-Gluconic acid δ-lactone, di(2-ethyl hexyl)phosphate, tributyl phosphate, dodecyl phosphate, 2-dimethylamino ethyl ester of (diphenyl methyl)-phosphoric acid, [2-(diphenyl phosphinyloxy)ethyl]trimethyl ammonium iodide, iodoacetate, fluoroacetate, Kinefusine, Australine, Castanospermine, Deoxynojirimycin, Deoxymannojirimycin, Swainsoninne, Mannostatin A, and the like.

In some embodiments of the compositions, methods, and uses described herein, a glycosylation inhibitor includes, but is not limited to, an inhibitor of N-linked glycan biosynthesis, including a mannosidase (e.g., a selective inhibitor of a mannosidase), an N-linked glycan N-acetylglucosaminyl transferase (e.g., a selective inhibitor of an N-linked glycan N-acetylglucosaminyl transferase), an N-linked glycan fucosyl transferase (e.g., a selective inhibitor of an N-linkedglycan fucosyl transferase), an N-linked glycan galactosyl transferase (e.g., a selective inhibitor of an N-linked glycan galactosyl transferase), an N-linkedglycan sialyl transferase (e.g., a selective inhibitor of an N-linked glycan sialyl transferase), an N-linked glycan sulfotransferase (e.g., a selective inhibitor of an N-linked glycan sulfotransferase), or an N-linked glycanglycophosphotransferase (e.g., a selective inhibitor of an N-linked glycanglycophosphotransferase) or a combination thereof. In some embodiments, the inhibitor of N-linked glycan biosynthesis is an inhibitor of late-stage N-linkedglycan biosynthesis (e.g., a selective inhibitor of late-stage N-linked glycan biosynthesis).

Use of inhibitors that block the modification reactions at different steps, can cause a cell to produce a glycoprotein, such as IgE, with altered carbohydrate structures. A number of alkaloid-like compounds have been identified that are specific inhibitors of the glucosidases and mannosidases involved in glycoprotein processing. These compounds cause the formation of glycoproteins with glucose-containing high mannose structures, or various high-mannose or hybrid chains, depending on the site of inhibition. (Elbein A D FASEB J. 1991 December; 5(15):3055-3063).

“Glycosylation inhibitor” or “glycosylation-inhibiting compound,” as the terms are used herein, refer to a substance or compound where a polypeptide or protein produced in the presence of the substance or compound comprises either at least one unglycosylated site or at least one carbohydrate moiety less at the same site than an otherwise identical polypeptide or protein which is produced by an otherwise identical cell under otherwise identical conditions but in the absence of the substance or compound. Glycosylation inhibitors include, but are not limited to, tunicamycin, tunicaymycin homologs, streptovirudin, mycospocidin, amphomycin, tsushimycin, antibiotic 24010, antibiotic MM 19290, bacitracin, corynetoxin, showdomycin, duimycin, 1-deoxymannonojirimycin, deoxynojirimycin, N-methyl-1-dexoymannojirimycin, brefeldin A, glucose and mannose analogs, 2-deoxy-D-glucose, 2-deoxyglucose, D-(+)-mannose, D-(+) galactose, 2-deoxy-2-fluoro-D-glucose, 1,4-dideoxy-1,4-imino-D-mannitol (DIM), fluoroglucose, fluoromannose, UDP-2-deoxyglucose, GDP-2-deoxyglucose, hydroxymethylglutaryl-CoA reductase inhibitors, 25-hydroxycholesterol, hydroxycholesterol, swainsonine, cycloheximide, puromycin, actinomycin D, monensin, m-Chlorocarbonyl-cyanide phenylhydrazone (CCCP), compactin, dolichyl-phosphoryl-2-deoxyglucose, N-Acetyl-D-Glucosamine, hypoxanthine, thymidine, cholesterol, glucosamine, mannosamine, castanospermine, glutamine, bromoconduritol, conduritol epoxide and conduritol derivatives, glycosylmethyl-p-nitrophenyltriazenes, .beta.-Hydroxynorvaline, threo-.beta.-fluoroasparagine, D-(+)-Gluconic acid.delta.-lactone, di(2-ethyl hexyl)phosphate, tributyl phosphate, dodecyl phosphate, 2-dimethylamino ethyl ester of (diphenyl methyl)-phosphoric acid, [2-(diphenyl phosphinyloxy)ethyl]trimethyl ammonium iodide, iodoacetate, and/or fluoroacetate. One of ordinary skill in the art will readily recognize or will be able to determine glycosylation-inhibiting substances that may be used in accordance with methods and compositions of the present invention without undue experimentation.

As used herein, in regard to the glycosylation inhibitors and IgE-specific glycosylation inhibitor agents described herein, “modulating” or “to modulate” generally means reducing or inhibiting the activity of IgE, as measured using a suitable in vitro, cellular or in vivo assay, such as those described herein in the Examples, by at least 5%, at least 10%, at least 25%, at least 50%, at least 60%, at least 70%, at least 80%, or 90% or more, compared to activity of the target or antigen in the same assay under the same conditions but without the presence of the inhibitor agents described herein. Specifically, activities of IgE that are modulated using the glycosylation inhibitors and IgE-specific glycosylation inhibitor agents described herein include binding of IgE to FcεRI or FcεRII, IgE half-life, regulating IgE secondary structure, mast cell activation, and/or triggering of anaphylaxis.

Australine is a polyhydroxylated pyrrolizidine alkaloid that is a good inhibitor of the alpha-glucosidase amyloglucosidase (50% inhibition at 5.8 microM), but does not inhibit beta-glucosidase, alpha- or beta-mannosidase, or alpha- or beta-galactosidase. Castanospermine is an indolizine alkaloid that inhibits alpha-glucosidase activities and alters glycogen distribution.

“Glycoside hydrolases,” “glycosidases,” or “glycosyl hydrolases” catalyze the hydrolysis of the glycosidic linkage to release smaller sugars. They are common enzymes with roles in nature including degradation of biomass such as cellulose and hemicellulose, in anti-bacterial defense strategies (e.g., lysozyme), in pathogenesis mechanisms (e.g., viral neuraminidases) and in normal cellular function (e.g., trimming mannosidases involved in N-linked glycoprotein biosynthesis). Together with glycosyltransferases, glycosidases form the major catalytic machinery for the synthesis and breakage of glycosidic bonds.

Several mannosidase processing inhibitors are known. Swainsonine, isolated from locoweed was shown to be a potent inhibitor of the Golgi mannosidase II, but to have no effect on mannosidase 1 (James, L. F., Elbein, A. D., Molyneux, R. J., and Warren, C. D. (1989) Swainsonine and related glycosidase inhibitors. Iowa State Press, Ames, Iowa). Deoxymannojirimycin is an inhibitor of rat liver mannosidase I. In intact cells, deoxymannojinimycin blocked the synthesis of complex types of N-linked oligosaccharides and caused the accumulation of glycoproteins having Man₇₋₉(GlcNAc)₂ structures, with Man₉(GlcNAc)₂ oligosaccharides predominating (Elbein, A. D., et al. (1984) Arch. Biochem. Biophys. 235, 579-588) Mannostatin A is a metabolite produced by the microorganism, Streptoverticillium verticillus, and this compound was reported to be an inhibitor of rat epididymal α-mannosidase. (Aoyagi, T., et al. (1989) J. Antibiot. 42, 883-889).

Kifunensine (Kif) was first isolated from the actinomycete, Kitasatosporia kifunense No. 9482 (M. Iwami, O. et al. J. Antibiot., 40, 612, 1987) and is a cyclic oxamide derivative of 1-amino-mannojirimycin. Kifunensine inhibits the animal Golgi enzyme, mannosidase I. When kifunensine is added to cultured mammalian cells at concentrations of 1 ug/ml or higher, it causes a complete shift in the structure of the N-linked oligosaccharides from complex chains to Man₉(GlcNAc)₂ structures, in keeping with its inhibition of mannosidase I.

Kifunensine has also shown promising immunomodulatory activity in α-mannosidase inhibition. The synthesis of kifunensine has been reported by both Fujisawa Pharmaceutical Co. (H. Kayakiri, et al., Tetrahedron Lett., 31, 225, 1990; H. Kayakiri, et al., Chem. Pharm. Bull., 39, 1392, 1991) and Hudlicky et al. (J. Rouden and T. Hudlicky, J. Chem. Soc. Perkin Trans. 1, 1095, 1993; J. Rouden, T. et al., J. Am. Chem. Soc., 116, 5099, 1994).

Treating cells with kifunensine (Kif) results in the inhibition of glycoprotein processing in those cells (Elbein et al (1991) FASEB J (5):3055-3063; and Bischoff et al (1990) J. Biol. Chem. 265(26):15599-15605). Kif blocks complex sugar attachment to modified proteins. However, if sufficient Kif is utilized to completely inhibit glycoprotein processing on lysosomal hydrolases, the resultant hydrolases have mannose-9 structures, which are not the most efficient substrates for the GlcNAc phosphotransferase enzyme.

Levels and/or types of complex carbohydrate structures can be measured using known methods, such as those described herein or in the Examples. For example, glycoproteins and their associated oligosaccharides can be characterized using endoglycosidases to differentiate between high mannose and complex type oligosaccharides (Maley et al (1989) Anal. Biochem. 180:195-204). Peptide-N.2.03.4-(N-acety-162-glucosaminyl)asparagine amidase (PNGaseF) is able to hydrolyze asparagines-linked (N-linked) oligosaccharides at the .beta.-aspartylglycosylamine bond to yield ammonia, aspartic acid and an oligosaccharide with an intact di-N-acetlychitobiose on the reducing end. The specificity of PNGase is broad because high mannose, hybrid, di-, tri- and tetraantennary complex, sulfated and polysialyl oligosaccharides are substrates. Additionally, endo-β-N-acetylglucosaminidase H (EndoH) effectively hydrolyzes the chitobiose unit in hybrid- and mannose-containing N-linked oligosaccharides possessing at three mannose residues, providing that the α-1,6-mannose arm has another mannose attached. Complex oligosaccharides are resistant to EndoH digestion.

To characterize the type of N-linked oligosaccharides present in glycoproteins, an aliquot of protein can be digested with PNGaseF (0.5% SDS, 1% β-mercaptoethanol, 50 mM NP-40, 50 mM Sodium Phosphate, pH 7.5) or EndoH (0.5% SDS, 1% β-mercaptoethanol, 50 mM Sodium Citrate, pH 5.5) under reducing conditions. The native and digested proteins are then analyzed by SDS-polyacrylamide electrophoresis under reducing conditions and the relative mobilities compared. If the glycoprotein contains only high mannose oligosaccharides the PNGaseF and EndoH treated samples will have a greater mobility than the untreated protein. The EndoH treated protein will have a slightly higher molecular weight due to the single remaining N-acetylglucosamine at each N-linked glycosylation site. If a glycoprotein contains only complex oligosaccharides, the EndoH treated protein will not have a shift in migration compared to the untreated protein. If there are both complex and high mannose oligosaccharides, then EndoH treated protein will be smaller than the non-treated glycoprotein but larger than the PNGaseF treated protein. The difference will be greater than that which can be accounted for by the remaining N-acetylglucosamine.

To generate simplified glycans on a target glycoprotein, such as IgE, endoglycosidases are used. An endoglycosidase is an enzyme that releases oligosaccharides from glycoproteins or glycolipids. Or it merely cleaves polysaccharide chains between residues that are not the terminal residue, although releasing oligosaccharides from conjugated protein and lipid molecules is more common. It breaks the glycosidic bonds between two sugar monomers in a polymer. Examples of endoglycosidases include, but are not limited to, endoglycosidase D, endoglycosidase F, endoglycosidaseF1, endoglycosidaseF2, endoglycosidase H, and endoglycosidase S.

“Glycoform” refers to a complex oligosaccharide structure comprising linkages of various carbohydrate units. Such structures are described in, e.g., Essentials of Glycobiology Varki et al., eds., Cold Spring Harbor Laboratory Press, Cold Spring Harbor, N.Y. (1999), which also provides a review of standard glycobiology nomenclature. Such glycoforms include, but are not limited to, G2, G1, G0, G-1, and G-2 (see, e.g., International Patent Publication Nos. WO98/58964 and WO 99/22764).

The term “glycan site occupancy” or “glycan occupancy” encompasses where a potential N-link or O-link glycosylation site in a protein can comprise a covalently linked carbohydrate moiety (i.e., the glycan site is occupied) or not (i.e., the glycan site is unoccupied). Where there are at least two potential glycosylation sites on a polypeptide, either none (0-glycan site occupancy), one (1-glycan site occupancy) or both (2-glycan site occupancy) can be occupied by a carbohydrate moiety.

“Glycosylation pattern” is defined as the pattern of carbohydrate units that are covalently attached to a protein (e.g., the glycoform) as well as to the site(s) to which the glycoform(s) are covalently attached to the peptide backbone of a protein, more specifically to an immunoglobulin protein.

By the term “glycosylation inhibiting amount” as the term is used herein is meant the amount of a substance or compound where a polypeptide or protein produced in the presence of the substance compound comprises a detectable decrease in glycosylation compared with an otherwise identical polypeptide or protein produced in the absence of the substance or compound. That is, either the polypeptide or protein comprises at least one more unglycosylated site (unoccupied glycan site) or comprises at least one carbohydrate moiety less at the same potential glycosylation site than an otherwise identical polypeptide or protein which is produced by a cell under otherwise identical conditions but in the absence of the substance or compound.

### Treatment of Allergic and Atopic Conditions

In some aspects, the methods described herein comprise administering an effective amount of the IgE-specific inhibitors or glycosylation inhibitors described herein to a subject having or diagnosed with an allergic disease or disorder.

As used herein, “allergy” shall refer to those inflammatory disorders caused by acquired hypersensitivity to a substance (allergen). Allergic conditions include eczema, allergic rhinitis or coryza, hay fever, bronchial asthma, urticaria (hives) and food allergies, and other atopic conditions. A “subject having an allergy” is a subject that has or is at risk of developing an allergic reaction in response to an allergen. An “allergen” refers to a substance that can induce an allergic or asthmatic response in a susceptible subject. The term “atopic” as used herein refers to a state of atopy or allergy to an allergen or a state of hypersensitivity to an allergen. Typically, atopic refers to Type I hypersensitivity which results from release of mediators (e.g., histamine and/or leukotrines) from IgE-sensitized basophils and mast cells after contact with an antigen (allergen). An example of atopic is atopic asthma, which is allergic asthma and is characterized by an IgE response.

Other atopic conditions suitable for use with the compositions and methods described herein include allergic rhinitis, gastrointestinal allergies, including food allergies, eosinophilia, conjunctivitis, and glomerulonephritis, as well as certain pathogen susceptibilities such as helminthic (e.g., leishmaniasis) and certain viral infections, including human immunodeficiency virus (HIV), and certain bacterial infections, including tuberculosis and lepromatous leprosy.

Allergens of interest include antigens found in food, such as strawberries, peanuts, milk polypeptides, egg whites, etc. Other allergens of interest include various airborne antigens, such as grass pollens, animal danders, house mite feces, etc. Molecularly cloned allergens include Dermatophagoides pteryonyssinus (Der P1); LoI pl-V from rye grass pollen; a number of insect venoms, including venom from jumper ant Myrmecia pilosula; Apis mellifera bee venom phospholipase A2 (PLA2 and antigen 5S; phospholipases from the yellow jacket Vespula maculifrons and white faced hornet Dolichovespula maculata; a large number of pollen polypeptides, including birch pollen, ragweed pollen, Parol (the major allergen of Parietaria officinalis) and the cross-reactive allergen Parjl (from Parietaria judaica), and other atmospheric pollens including Olea europaea, Artemisia sp., gramineae, etc. Other allergens of interest are those responsible for allergic dermatitis caused by blood sucking arthropods, e.g. Diptera, including mosquitos (Anopheles sp., Aedes sp., Culiseta sp., Culex sp.); flies (Phlebotomus sp., Culicoides sp.) particularly black flies, deer flies and biting midges; ticks (Dermacenter sp., Ornithodoros sp., Otobius sp.); fleas, e.g. the order Siphonaptera, including the genera Xenopsylla, Pulex and Ctenocephalides felis. The specific allergen may be a polysaccharide, fatty acid moiety, polypeptide, etc.

Asthma refers to a chronic inflammatory disease of the respiratory system in which the airway occasionally constricts, becomes inflamed, and is lined with excessive amounts of mucus, often in response to one or more triggers. Asthma can be defined simply as reversible airway obstruction in an individual over a period of time. Asthma can be allergic/atopic or non-allergic. Asthma is characterized by the presence of cells such as eosinophils, mast cells, basophils, and activated T lymphocytes in the airway walls. With chronicity of the process, secondary changes occur, such as thickening of basement membranes and fibrosis. The disease is characterized by increased airway hyperresponsiveness to a variety of stimuli, and airway inflammation and constriction. This airway narrowing causes symptoms such as wheezing, shortness of breath, chest tightness, and coughing. The airway constriction responds to bronchodilators. Between episodes, most patients feel well but can have mild symptoms and they can remain short of breath after exercise for longer periods of time than the unaffected individual. The symptoms of asthma can range from mild to life threatening.

Asthma can be triggered by such things as exposure to an allergen (allergic asthma), or non-allergens (non-allergic asthma) such as cold air, pollution (e.g., ozone), warm air, moist air, exercise or exertion, or emotional stress. In children, the most common triggers are viral illnesses such as those that cause the common cold (Zhao J., et. al., 2002, J Pediatr. Allergy Immunol. 13: 47-50).

Common allergens that trigger the allergic asthma include “seasonal” pollens, year-round dust mites, molds, pets, and insect parts, foods, such as fish, egg, peanuts, nuts, cow's milk, and soy, additives, such as sulfites, work-related agents, such as latex. Approximately 80% of children and 50% of adults with asthma also have allergies.

Common irritants that can trigger asthma in airways that are hyperreactive include respiratory infections, such as those caused by viral “colds,” bronchitis, and sinusitis, medication drugs, such as aspirin, other NSAIDs (nonsteroidal antiinflammatory drugs), and beta blockers (used to treat blood pressure and other heart conditions), tobacco smoke, outdoor factors such as ozone, smog, weather changes, and diesel fumes; indoor factors such as paint, detergents, deodorants, chemicals, and perfumes; nighttime GERD (gastroesophageal reflux disorder); exercise, especially under cold dry conditions; work-related factors such as chemicals, dusts, gases, and metals; emotional factors, such as laughing, crying, yelling, and distress; and hormonal factors, such as in premenstrual syndrome.

Regardless of the trigger, asthma is associated with reversible airway obstruction and airway hyperreactivity (AHR), an increased sensitivity of the airways to nonspecific stimuli such as cold air or respiratory irritants, and can be quantitated by responsiveness to methacholine or histamine. A patient diagnosed as asthmatic will generally have multiple indications over time, including wheezing, asthmatic attacks, and a positive response to methacholine challenge, i.e., a PC20 on methacholine challenge of less than about 4 mg/ml. The basic diagnosis and measurement of asthma is peak flow rates and the following diagnostic criteria are used by the British Thoracic Society (Pinnock H., and Shah R., 2007, Br. Med. J. 334 (7598): 847-50): ≧20% difference on at least three days in a week for at least two weeks; ≧20% improvement of peak flow following treatment, for example: 10 minutes of inhaled β-agonist (e.g., salbutamol), six week of inhaled corticosteroid (e.g., beclometasone), and 14 days of 30 mg prednisolone; and ≧20% decrease in peak flow following exposure to a trigger (e.g., exercise). Further guidelines for diagnosis may be found, for example, in the National Asthma Education Program Expert Panel Guidelines for Diagnosis and Management of Asthma, National Institutes of Health, 1991, Pub. No. 91-3042.

The term “allergic respiratory disorder” or “hypersensitivity disease” refers to allergic diseases and/or disorders of the lungs or respiratory system. Allergic disorders are characterized by hypersensitivity to an allergen.

As used herein, “airway hyperreactivity” refers to the narrowing of air passages of the lungs (“airways”) in response to stimuli such as pollen, grains in the air, changes of temperature, emotional shock, or exercise. Airway hyperreactivity (AHR) is a cardinal feature of asthma, and is observed in all forms of asthma, including asthma induced with allergen and non-allergen such as ozone exposure.

The term “COPD” is generally applied to chronic respiratory disease processes characterized by the persistent obstruction of bronchial air flow. Typical COPD patients are those suffering from conditions such as bronchitis, cystic fibrosis, asthma or emphysema.

The term “allergic rhinitis” as used herein is characterized by any of the following symptoms: obstruction of the nasal passages, conjuctival, nasal and pharyngeal itching, lacrimation, sneezing, or rhinorrhea. These symptoms usually occur in relationship to allergen exposure.

The methods described herein, in some embodiments, comprise administering an effective amount of the IgE-specific inhibitors or glycosylation inhibitors described herein to a subject in order to alleviate a symptom of an allergic or atopic disease or disorder. As used herein, “alleviating a symptom of an allergic or atopic disease or disorder” is ameliorating any condition or symptom associated with the allergic or atopic disease, such as. As compared with an equivalent untreated control, such as a subject prior to the administration of the IgE-specific inhibitors or glycosylation inhibitors, such reduction or degree of prevention is at least 5%, 10%, 20%, 40%, 50%, 60%, 80%, 90%, 95%, or more as measured by any standard technique known to one of ordinary skill in the art. A patient or subject who is being treated for an allergic or atopic disease is one who a medical practitioner has diagnosed as having such a condition. Diagnosis can be by any suitable means. Diagnosis and monitoring can involve, for example, detecting or monitoring for a symptom of asthma, such as airway epithelium injury, airway smooth muscle spasm or airway hyperresponsiveness, airway mucosa edema, increased mucus secretion, excessive, T cell activation, or desquamation, atelectasis, cor pulmonale, pneumothorax, subcutaneous emphysema, dyspnea, coughing, wheezing, shortness of breath, tachypnea, fatigue, decreased forced expiratory volume in the 1st second (FEV₁), arterial hypoxemia, respiratory acidosis, inflammation, including unwanted elevated levels of mediators such as IL-4, IL-5, IgE, histamine, substance P, neurokinin A, calcitonin gene-related peptide or arachidonic acid metabolites such as thromboxane or leukotrienes (LTD₄ or LTC₄), and cellular airway wall infiltration, e.g., by eosinophils, lymphocytes, macrophages or granulocytes.

The terms “subject,” “patient,” and “individual” as used in regard to any of the methods described herein are used interchangeably herein, and refer to an animal, for example a human, recipient of the inhibitos described herein. For treatment of disease states which are specific for a specific animal such as a human subject, the term “subject” refers to that specific animal. The terms “non-human animals” and “non-human mammals” are used interchangeably herein, and include mammals such as rats, mice, rabbits, sheep, cats, dogs, cows (bovine), pigs, horses, opossums, platypus, and non-human primates, such as rhesus macaque. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, cow (bovine), pig, and the like. A production mammal, e.g. cow, sheep, pig, and the like are also encompassed in the term subject.

As used herein, in regard to any of the compositions, methods, and uses comprising glycosylation or IgE-specific inhibitors described herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a an allergic or atopic condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “effective amount” as used herein refers to the amount of a glycosylation or IgE-specific inhibitor described herein, needed to alleviate at least one or more symptom of the disease or disorder being treated, and relates to a sufficient amount of pharmacological composition to provide the desired effect, i.e., reverse the functional exhaustion of antigen-specific T cells in a subject having a chronic immune condition, such as cancer or hepatitis C. The term “therapeutically effective amount” therefore refers to an amount of the glycosylation or IgE-specific inhibitors described herein, using the methods as disclosed herein, that is sufficient to provide a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions, methods, and uses that exhibit large therapeutic indices are preferred. A therapeutically effective dose can be estimated initially from cell culture assays. Also, a dose can be formulated in animal models to achieve a circulating plasma concentration range that includes the IC50, which achieves a half-maximal inhibition of measured function or activity) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The glycosylation or IgE-specific inhibitors described herein can be administered to a subject in need thereof by any appropriate route which results in an effective treatment in the subject. As used herein, the terms “administering,” and “introducing” are used interchangeably and refer to the placement of a glycosylation or IgE-specific inhibitor into a subject by a method or route which results in at least partial localization of such agents at a desired site, such as a site of inflammation, such that a desired effect(s) is produced.

In some embodiments, the glycosylation or IgE-specific inhibitors described herein can be administered to a subject having an allergic or atopic condition by any mode of administration that delivers the agent systemically or to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the glycosylation or IgE-specific inhibitors, other than directly into a target site, tissue, or organ, such that it enters the subject's circulatory system and, thus, is subject to metabolism and other like processes.

For the clinical use of the methods described herein, administration of the glycosylation or IgE-specific inhibitors described herein, can include formulation into pharmaceutical compositions or pharmaceutical formulations for parenteral administration, e.g., intravenous; mucosal, e.g., intranasal; ocular, or other mode of administration. In some embodiments, the glycosylation or IgE-specific inhibitors described herein, can be administered along with any pharmaceutically acceptable carrier compound, material, or composition which results in an effective treatment in the subject. Thus, a pharmaceutical formulation for use in the methods described herein can contain a glycosylation or IgE-specific inhibitor or combination thereof, as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, a glycosylation or IgE-specific inhibitor. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alchols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

The glycosylation or IgE-specific inhibitors described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (79) nasally. Additionally, a bispecific or multispecific polypeptide agent can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Further embodiments of the formulations and modes of administration of the compositions comprising glycosylation or IgE-specific inhibitors described herein, that can be used in the methods described herein are described below.

Parenteral Dosage Forms.

Parenteral dosage forms of the glycosylation or IgE-specific inhibitors can also be administered to a subject with a chronic immune condition by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol Formulations.

A glycosylation or IgE-specific inhibitor can be packaged in a pressurized aerosol container together with suitable propellants, for example, hydrocarbon propellants like propane, butane, or isobutane with conventional adjuvants. A glycosylation or IgE-specific inhibitor described herein, can also be administered in a non-pressurized form such as in a nebulizer or atomizer. A glycosylation or IgE-specific inhibitor or combinations thereof described herein, can also be administered directly to the airways in the form of a dry powder, for example, by use of an inhaler.

Suitable powder compositions include, by way of illustration, powdered preparations of a glycosylation or IgE-specific inhibitor described herein, thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the A glycosylation or IgE-specific inhibitors described herein, further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms.

In some embodiments of the aspects described herein, a glycosylation or IgE-specific inhibitor described herein, can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of a compound of formula (I) is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the glycosylation or IgE-specific inhibitors or combinations thereof described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm & Haas, Spring House, Pa. USA).

In some embodiments of the methods described herein, a glycosylation or IgE-specific inhibitor described herein, for use in the methods described herein is administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of a glycosylation or IgE-specific inhibitor), or combinations thereof described herein, administered over the course of treatment to the subject or patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:
1. A method of inhibiting or blocking functional activity of IgE comprising administering to a subject in need thereof an inhibitor of glycosylation or an IgE-specific glycosylation inhibitor.
2. A method of treating an allergic or atopic disease in a subject in need thereof comprising administering to a subject in need thereof an inhibitor of glycosylation or an IgE-specific glycosylation inhibitor, wherein said inhibitor blocks functional activity of IgE, thereby treating an allergic or atopic disease.
3. The method of any one of paragraphs 1 or 2, wherein the IgE-specific glycosylation inhibitor blocks or inhibits glycosylation at the glycosylation sequon at N394 of IgE.
4. The method of paragraph 3, wherein the IgE-specific glycosylation inhibitor blocks or inhibits the glycosylation sequon at N394 of IgE and prevents or inhibits IgE-mediated signaling.
5. The method of any one of paragraphs 3 or 4, wherein the IgE-specific inhibitor is an antibody or antigen-binding fragment thereof that specifically binds or is directed against the glycosylation sequon at N394 of IgE.
6. The method of any one of paragraphs 3 or 4, wherein the IgE-specific inhibitor is an antibody or antigen-binding fragment thereof that specifically binds or is directed against a site on a target in the proximity of the glycosylation sequon at N394 of IgE, thereby providing steric hindrance for the interaction of IgE with its receptor.
7. The method of any one of paragraphs 3-5, wherein the antibody or antigen-binding fragment thereof that specifically binds or is directed against the glycosylation sequon at N394 of IgE, does not bind to IgE when bound to either of the Fcε receptors.
8. The method of any one of paragraphs 1-4, wherein the IgE-specific inhibitor is a small molecule compound that binds to the glycosylation sequon at N394 of IgE, thereby inhibiting or preventing IgE binding to either of the Fcε receptors.
9. The method of paragraph 8, wherein the small molecule inhibitor specifically binds the glycosylation sequon at N394 of IgE when glycosylated or specifically binds the glycosylation sequon at N394 of IgE when unglycosylated, and prevents the glycosylation at N394.
10. The method of any one of paragraphs 1 or 2, wherein the glycosylation inhibitor is selected from tunicamycin, a tunicaymycin homolog, streptovirudin, mycospocidin, amphomycin, tsushimycin, antibiotic 24010, antibiotic MM 19290, bacitracin, corynetoxin, showdomycin, duimycin, 1-deoxymannonojirimycin, deoxynojirimycin, N-methyl-1-dexoymannojirimycin, brefeldin A, a glucose analog, a mannose analog, 2-deoxy-D-glucose, 2-deoxyglucose, D-(+)-mannose, D-(+) galactose, 2-deoxy-2-fluoro-D-glucose, 1,4-dideoxy-1,4-imino-D-mannitol (DIM), fluoroglucose, fluoromannose, UDP-2-deoxyglucose, GDP-2-deoxyglucose, a hydroxymethylglutaryl-CoA reductase inhibitor, 25-hydroxycholesterol, hydroxycholesterol, swainsonine, cycloheximide, puromycin, actinomycin D, monensin, m-Chlorocarbonyl-cyanide phenylhydrazone (CCCP), compactin, dolichyl-phosphoryl-2-deoxyglucose, N-Acetyl-D-Glucosamine, hypoxanthine, thymidine, cholesterol, glucosamine, mannosamine, castanospermine, glutamine, bromoconduritol, conduritol epoxide, a conduritol derivative, aglycosylmethyl-p-nitrophenyltriazene, β-Hydroxynorvaline, threo-β-fluoroasparagine, D-(+)-Gluconic acid δ-lactone, di(2-ethyl hexyl)phosphate, tributyl phosphate, dodecyl phosphate, 2-dimethylamino ethyl ester of (diphenyl methyl)-phosphoric acid, [2-(diphenyl phosphinyloxy)ethyl]trimethyl ammonium iodide, iodoacetate, fluoroacetate, Kinefusine, Australine, Castanospermine, Deoxynojirimycin, Deoxymannojirimycin, Swainsoninne, and Mannostatin A.
11. The method of any one of paragraphs 1 or 2, wherein the glycosylation inhibitor is selected from an inhibitor of N-linked glycan biosynthesis, an inhibitor of N-linked glycan N-acetylglucosaminyl transferase, an inhibitor of N-linked glycan fucosyl transferase, an inhibitor of N-linked glycan galactosyl transferase, an inhibitor of N-linkedglycan sialyl transferase, an inhibitor of N-linked glycan sulfotransferase, an inhibitor of N-linked glycanglycophosphotransferase, or a combination thereof.
12. The method of any one of paragraphs 1 or 2, wherein the glycosylation inhibitor is not EndoS.
13. The method of any one of paragraphs 2-12, wherein the allergic or atopic disorder is selected from allergic asthma, eczema, allergic rhinitis or coryza, hay fever, bronchial asthma, urticaria (hives), gastrointestinal allergies, eosinophilia, conjunctivitis, and glomerulonephritis.

### Examples

Immunoglobulin ε (IgE) is important for resistance to parasitic infections^{6,7} and protection against venom toxins^{8,9}. Yet IgE is also responsible for triggering allergic reactions, which affect over 20% of people worldwide¹. Allergic reactions can manifest as localized irritation, asthma, or life-threating anaphylaxis in extreme cases. IgE is the least abundant immunoglobulin in circulation, with a short serum half-life of 2 days. However, IgE can persist for weeks bound to the surface of mast cells in tissues, basophils in circulation, and dendritic cells, by the high affinity IgE receptor, FcεRI¹⁰. Exposure to allergen recognized by cell-bound IgE crosslinks FcεRI and promotes mast cell activation, degranulation, and release of mediators including histamine, prostaglandins, and leukotrienes, which are responsible for allergic symptoms, including vasodilation, vascular permeability, and smooth muscle contractility^{10,11}. Various mediators in this cascade have been targeted, with some therapeutic benefits, after onset of symptoms. Recently developed preventive therapies neutralize circulating IgE or deplete IgE-producing cells have demonstrated some efficacy in the treatment of allergic asthma and rhinitis¹¹⁻¹³.

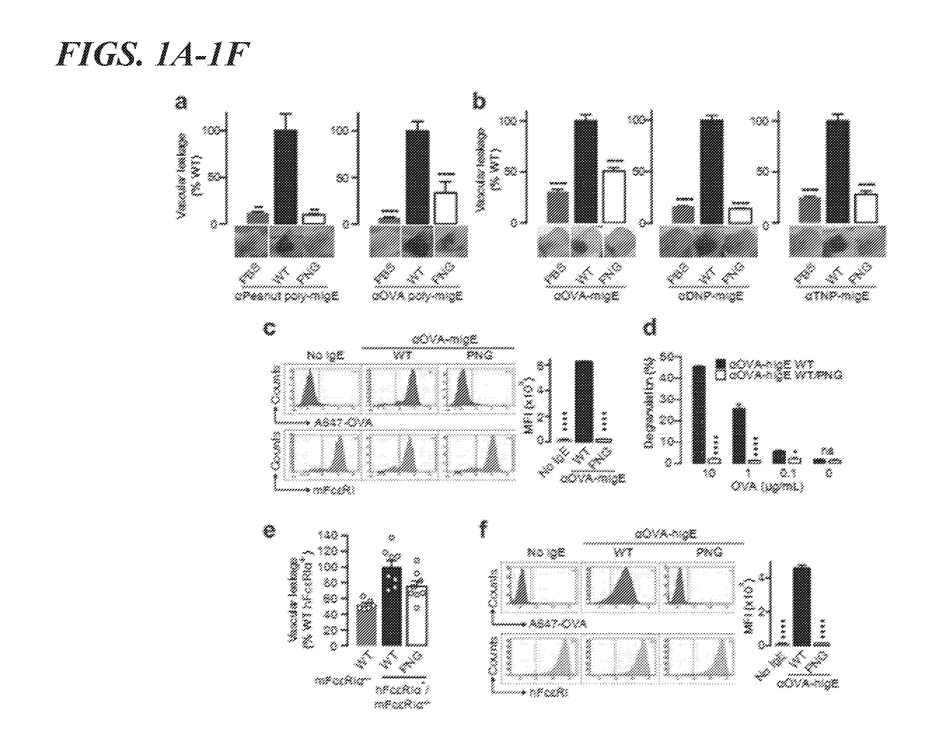

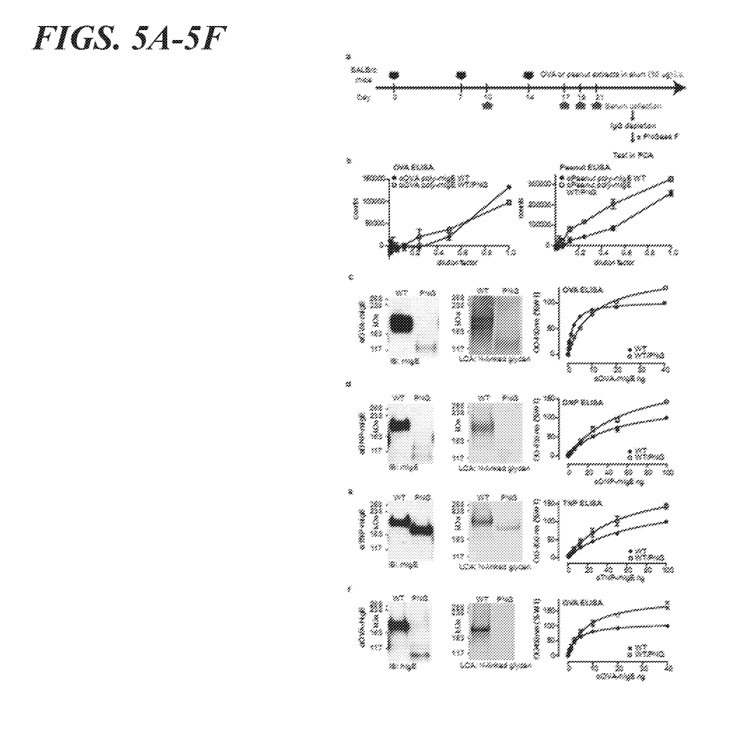

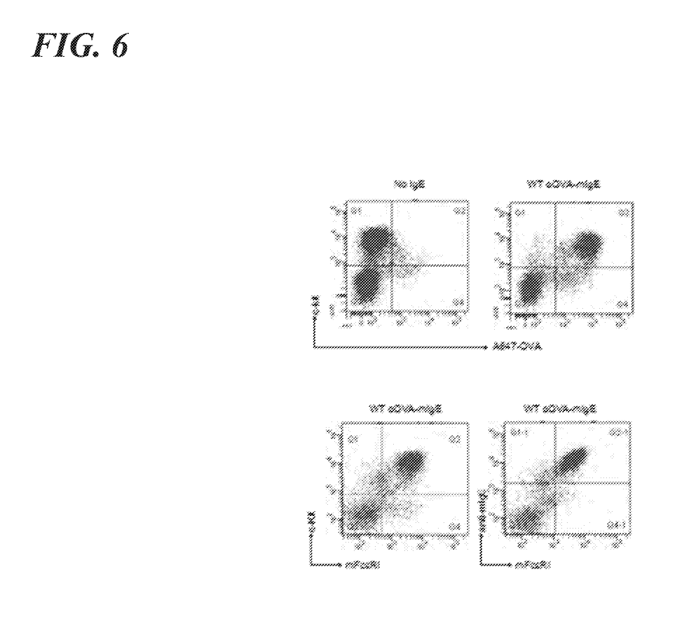

IgE is the most heavily glycosylated monomeric immunoglobulin in mammals, with seven N-linked glycosylation consensus sequences (N-X-S/T) distributed across the constant chains of human IgE⁵. Although glycosylation is documented to have profound effects on IgG and IgA, the contribution of glycosylation to IgE biology is unknown^{5,14}. Therefore, we sought to determine whether glycosylation was required for the in vivo activity of IgE. We enriched polyclonal mouse IgE (poly-mIgE) specific for ovalbumin (OVA) or extracts from the common food allergen, peanuts, from the serum of immunized mice, and removed all N-linked glycans by treatment with the endoglycosidase, Peptide-N-Glycosidase F (PNG; FIG. 5A). PNG-treatment did not reduce recognition of OVA or peanut extracts by poly-mIgE in an ELISA assay (FIG. 5B). Employing a model of passive cutaneous anaphylaxis (PCA) highly dependent on the interactions between IgE, FcεRI⁺ mast cells, and allergens¹⁵, we intradermally injected poly-mIgE in the ears of mice, and challenged the mice the next day with appropriate allergens and Evan's blue dye intravenously. Poly-mIgE elicited robust anaphylaxis to OVA or peanut extracts, as measured by vascular leakage (FIG. 1A). PNG-poly-mIgE, enzymatically stripped of all N-linked glycans, displayed significantly compromised anaphylactic responses to both allergens. Next, we treated three monoclonal mIgEs specific for model allergens OVA, dinitrophenol (DNP), or trinitrophenol (TNP) with PNG. Enzymatic de-glycosylation and antigen binding was verified by lectin blotting and ELISA assays (FIGS. 5C-5E). While mIgE specific for OVA, DNP, or TNP triggered strong allergen-specific anaphylactic responses in vivo, PNG-treatment significantly attenuated IgE-mediated inflammation (FIG. 1B). Allergic reactions are highly dependent on IgE and FcεRI interactions^{2,10,15}. To determine the contribution of glycosylation to interactions with mouse FcεRI (mFcεRI), murine bone marrow-derived mast cells (mBMMCs) were sensitized in vitro with αOVA-mIgE or PNG-αOVA-mIgE overnight, and mFcεRI-bound mIgE was detected by Alexa647-OVA (A647-OVA). Flow Cytometry revealed while αOVA-mIgE bound to the mast cells, PNG-αOVA-mIgE did not (FIG. 1C, FIG. 6). These results demonstrate that N-linked glycans on mIgE are necessary for mast cell binding and eliciting anaphylaxis to multiple antigens in vivo.

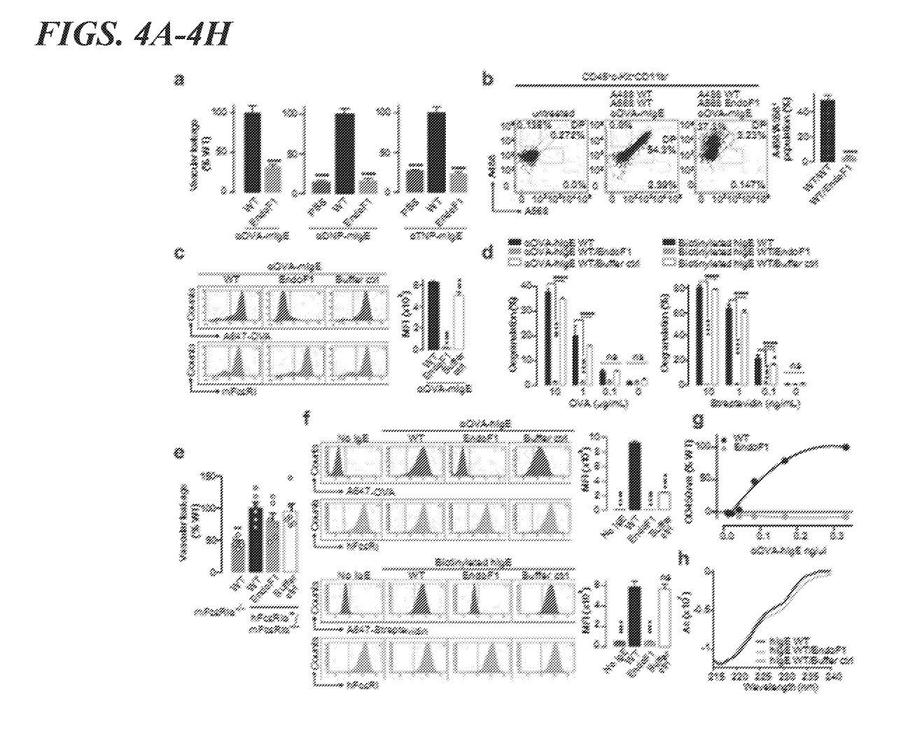

We next generated and enzymatically de-glycosylated OVA-specific human IgE (αOVA-hIgE, PNG-αOVA-hIgE, FIG. 4F). Human LAD2 mast cells¹⁶ were sensitized in vitro with αOVA-hIgE or PNG-αOVA-hIgE. OVA stimulation resulted in dose-dependent degranulation of αOVA-hIgE-sensitized mast cells, as assessed by β-hexosaminidase release. However, PNG-αOVA-hIgE was incapable of instigating degranulation upon OVA-stimulation (FIG. 1D). In parallel, we administered αOVA-hIgE or PNG-αOVA-hIgE to transgenic mice expressing human FcεRI that lacked mFcεRI (hFcεRI/mFcεRI^{-/-})¹⁷. αOVA-hIgE was unable to elicit PCA following OVA administration in mFcεRI^{-/-} mice, but triggered robust vascular leakage in the ears of hFcεRI/mFcεRI^{-/-} mice (FIG. 1E). The response was muted in PNG-αOVA-hIgE treated ears (FIG. 1E), confirming N-linked glycosylation is also essential for the in vivo activity of human IgE.

To determine whether hIgE glycosylation was important for hFcεRI binding, HeLa cells engineered to express hFcεRI (hFcεRI⁺-HeLa) were sensitized with αOVA-hIgE, incubated with Alexa-647-OVA (A647-OVA), and analyzed by Flow Cytometry. αOVA-hIgE was bound by hFcεRI⁺-HeLa cells, as detected by A647-OVA. However, no A647-OVA was detected when cells were sensitized with PNG-αOVA-hIgE (FIG. 1F), despite retaining OVA recognition as determined by ELISA (FIG. 5F). Together, these results indicate that IgE glycosylation is essential for the initiation of anaphylaxis and interactions with FcεRI.

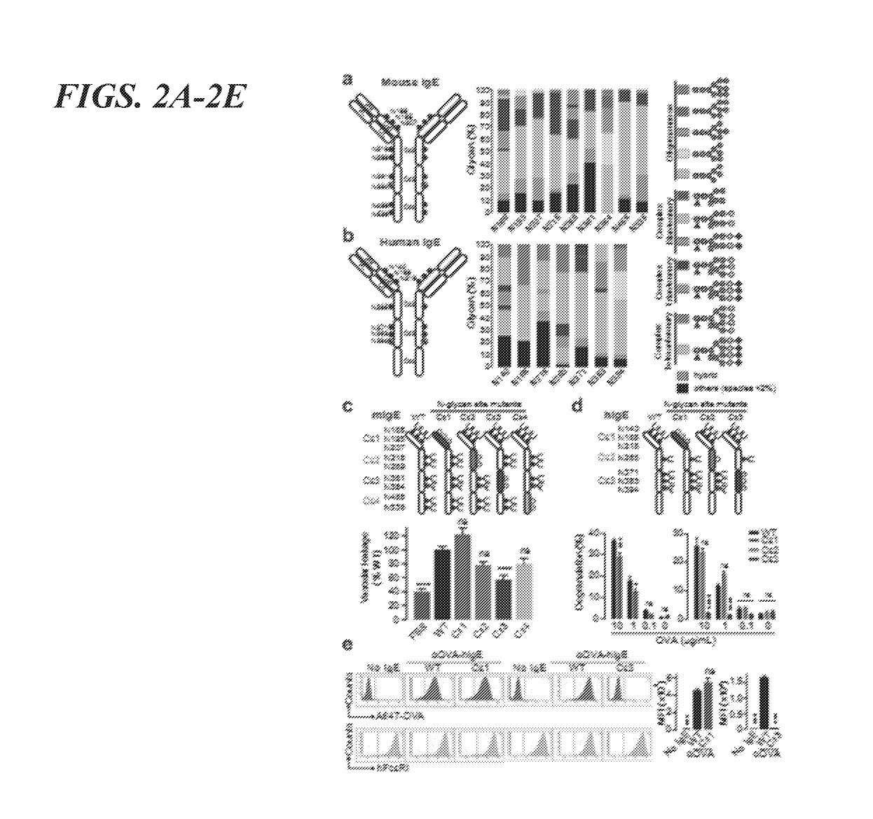

Next, we sought to analyze site-specific glycosylation throughout the four constant domains (CE1-4) of mouse and human aOVA-IgE by glycopeptide mass spectroscopy. Eight of nine N-linked glycosylation consensus sequences (N-X-S/T) in aOVA-mIgE were primarily occupied by highly processed complex biantennary glycans (FIG. 2A). Similarly, all but one site on aOVA-hIgE contained predominantly complex antennary structures (FIG. 2B). Interestingly, a single site in CE3 of mIgE and hIgE was occupied by an oligomannose structure (FIG. 2A, N384 and FIG. 2B, N394, respectively), consistent with previous reports of hIgE glycosylation¹⁸⁻²⁰.

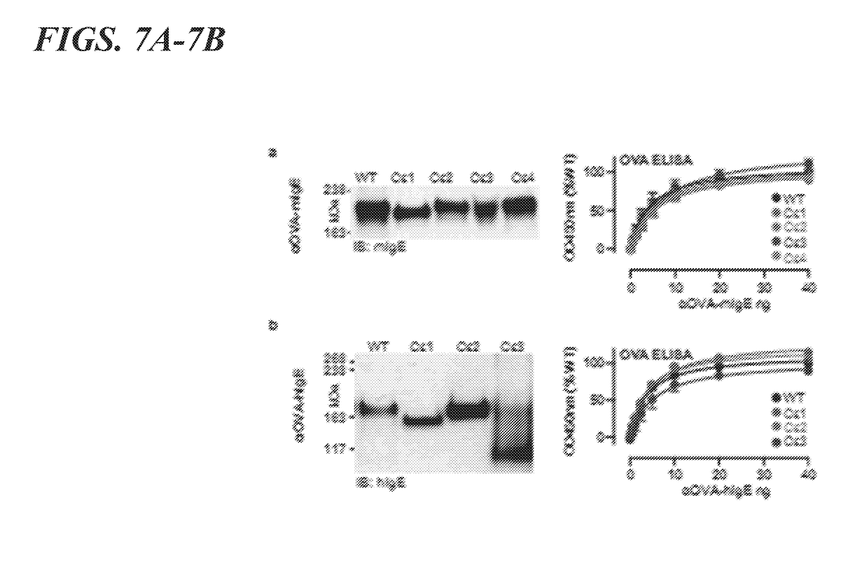

To map the glycan requirements of IgE, we generated a panel of aOVA-mIgE mutants selectively lacking all glycosylation sites on each CE1, CE2, CE3, or CE4 domain, by mutating asparagine (N) to glutamine (Q). These domain-specific mutants recognized OVA similarly (FIG. 7A), and were tested in vivo by PCA. WT-, CE1-, CE2-, or CE4-aOVA-mIgE domain mutants promoted robust vascular leakage following OVA challenge (FIG. 2C). However, anaphylaxis elicited by the CE3-aOVA-mIgE mutant was significantly attenuated. aOVA-hIgE domain-specific glycosylation mutants were generated, found to recognize OVA similarly to WT-aOVA-hIgE (FIG. 7B), and were tested for their ability to activate mast cells (FIG. 2D). Although WT- and CE2-aOVA-hIgE domain mutants triggered robust degranulation upon OVA stimulation, degranulation was slightly reduced in mast cells sensitized with CE1-aOVA-hIgE. However, mutation of CE3 glycosylation sites completely abolished OVA-specific degranulation. While CE1-aOVA-hIgE mutants bound to FcεRI⁺-HeLa cells as determined by A647-OVA Flow Cytometry, CE3-aOVA-hIgE did not (FIG. 2E). Together, these results suggest that CE3 glycosylation is required for both mouse and human IgE to bind FcεRI and initiate anaphylaxis.

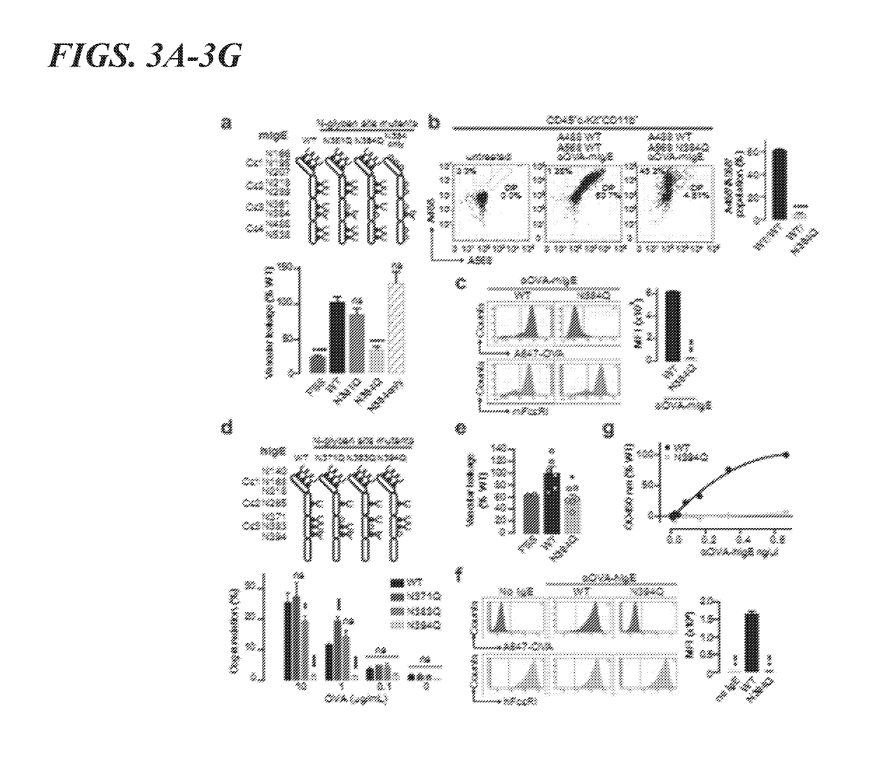

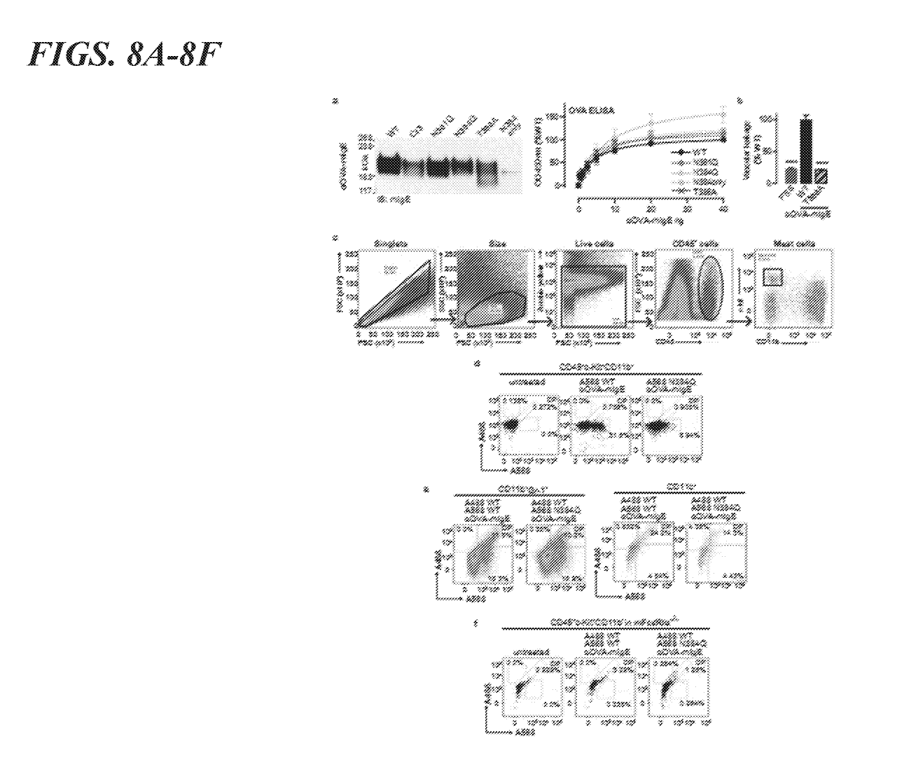

Two N-linked glycosylation sites are present in CE3 of mIgE. To dissect the role of these individual sites, we mutated N361 or N384 to Q. Both WT- and N361Q-aOVA-mIgE elicited a robust PCA reaction in vivo. However, N384Q-aOVA-mIgE was incapable of initiating anaphylaxis (FIG. 3A), despite being able to recognize OVA (FIG. 8A). Moreover, a reciprocal mutant, in which all N-linked glycosylation sites were disrupted except N384 (N384only-aOVA-mIgE), promoted vigorous PCA upon OVA administration (FIG. 3A, FIG. 8A). To confirm the importance of N384 glycosylation for mIgE function, the glycosylation consensus sequence was disrupted by mutation of the third-position amino acid (T386A). Indeed, T386A-aOVA-mIgE was unable to initiate anaphylaxis (FIG. 8B). Collectively, our data reveals that glycosylation specifically at N384 in the CE3 domain of mIgE is essential for initiation of anaphylaxis.

We hypothesized that N384 was required for mIgE binding to FcεRI on mast cells. To test this in vivo, equal amounts of A488- and A568-labeled WT-aOVA-mIgE (A488-WT/A568-WT) or A488-WT-aOVA-mIgE and A568-N384Q-aOVA-mIgE (A488-WT/A568-N384Q) were injected intradermally into mouse ears, and assessed the next day by Flow Cytometry (FIG. 3B, FIG. 8C). Dermal mast cells (CD45⁺ c-Kit⁺ CD11b⁻) bound WT-aOVA-mIgE regardless of fluorophore conjugate, but preferentially bound WT- over N384Q-aOVA-mIgE when both were administered. No N384Q-aOVA-mIgE binding was observed when administered singly (FIG. 8D). Murine phagocytes (CD11b⁺, CD11c⁺) do not express FcεRI, but did interact with both WT- and N384-aOVA-mIgE (FIG. 8E). Dermal mast cells from mFcεRI^{-/-} mice showed minimal binding of aOVA-mIgE (FIG. 8F). We then loaded mBMMCs with WT- or N384Q-aOVA-mIgE, and detected A647-OVA bound to WT- but not N384Q-aOVA-mIgE sensitized cells (FIG. 3C). These results identify the N384 glycan as essential for stable mIgE interactions with FcεRI on mast cells in vitro and in vivo.

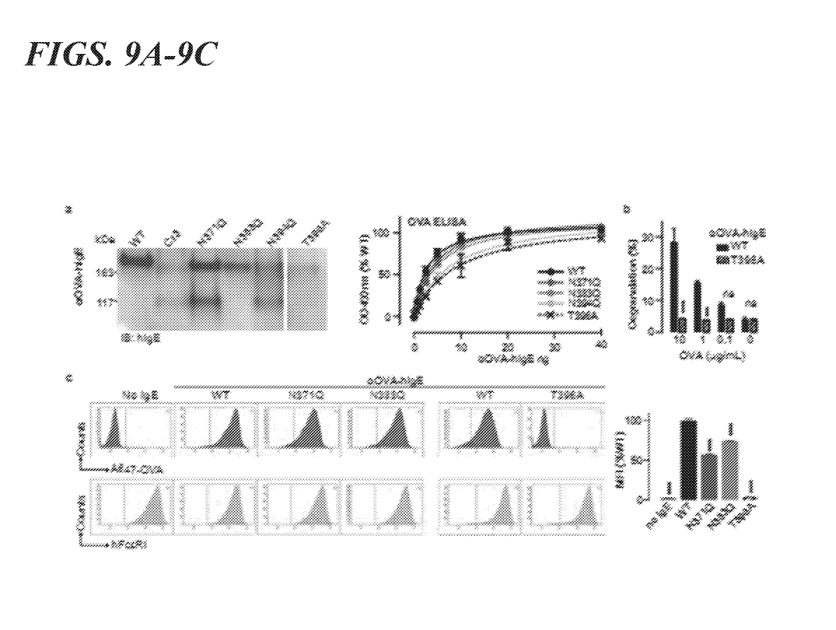

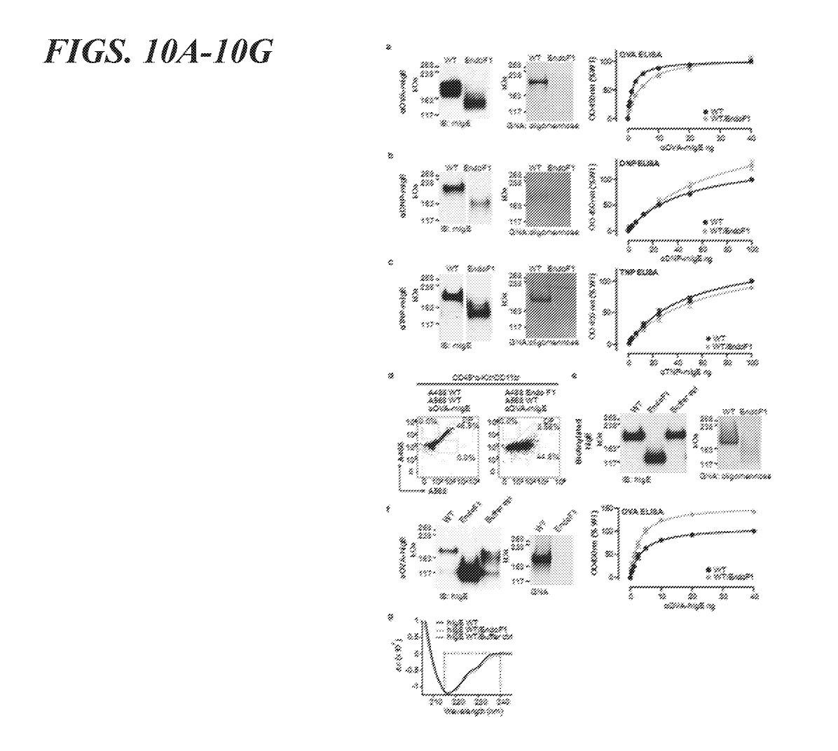

Three N-linked glycosylation sites are occupied in CE3 of aOVA-hIgE (FIG. 2B). To determine the contribution of these sites to the initiation of anaphylaxis, individual hIgE CE3 glycosylation site mutants were generated, and examined for mast cell degranulating activity. N371Q- or N383Q-aOVA-hIgE exhibited slightly altered ability to activate mast cells upon OVA-stimulation. However, mutation of either the first position (N394Q-aOVA-hIgE) or third position (T396A-aOVA-hIgE) of the N394 site ablated OVA-mediated degranulation (FIG. 3D, FIGS. 9A-9B). Further, N394Q-aOVA-hIgE was unable to elicit PCA in hFcεRI⁺/mFcεRI^{-/-} mice (FIG. 3E). We next examined the role of this glycan at N394 in hFcεRI binding. Flow Cytometry of hFcεRI⁺ HeLa cells primed with WT-, N371Q-, N383Q-, N394Q-, or T396A-aOVA-hIgE and treated with A647-OVA revealed that WT-, N371Q-, N383Q- but not N394Q- or T396A-aOVA-hIgE bound hFcεRI (FIG. 3F, FIG. 9C). Further, while WT-aOVA-hIgE bound and saturated the hFcεRI a-chain in vitro, N394Q-aOVA-hIgE did not (FIG. 3G). These results identify the glycosylation sites N394 and N384 in hIgE and mIgE, respectively, as essential for interacting with FcεRI and mast cells, and are required for initiating allergic inflammation.

hIgE N394 and mIgE N384 are occupied by oligomannose glycans, while predominantly complex, antennary glycans occur at all other sites in aOVA-IgE (FIGS. 2A-2B). Endoglycosidase F1 (EndoF1) cleaves between GlcNAc residues on N-linked oligomannose and hybrid glycans, leaving complex glycans unaffected. Thus, we treated mIgE specific for OVA, DNP, or TNP with EndoF1 (FIGS. 9A-9C), and tested these mIgE preparations in vivo by PCA. Selective removal of oligomannose glycans by EndoF1 significantly attenuated anaphylaxis, compared to WT-mIgE (FIG. 4A). Furthermore, EndoF1-aOVA-mIgE did not bind to mast cells in vivo or in vitro (FIGS. 4B-4C, FIG. 9D). Together, these results indicate that the oligomannose glycan on mIgE is essential for initiating anaphylaxis and interacting with mast cells.

Next, aOVA-hIgE or biotinylated hIgE was treated with EndoF1 (FIGS. 9E-9F). This abolished the ability of hIgE to activate mast cells following crosslinking by OVA or streptavidin (FIG. 4D), and reduced PCA-mediated vascular leakage in vivo in hFcεRI⁺/mFcεRI^{-/-} mice (FIG. 4E). Further, EndoF1-treatment ablated binding to hFcεRI, as determined by Flow Cytometry and saturation binding experiments (FIGS. 4F-4G). Studies have shown that removal of the single N-linked glycan on IgG Fc results in a conformation change that prevents FcyR binding²¹. Thus, the contribution of the oligomannose glycan to hIgE secondary structure was examined by circular dichroism²² (CD). While CD spectra of WT- and EndoF1-buffer control hIgE overlapped, a shift was observed following EndoF1 treatment, reflecting small changes in the overall secondary structure of hIgE (FIG. 4H, FIG. 8G). Together, these results indicate that a local structural alteration by oligomannose-removal is responsible for modifying hIgE biology.

Herein, we demonstrate that glycosylation of IgE is an absolute requirement for initiation of the allergic cascade. This requirement was mapped to a single N-linked site in the CE3 domain of mouse and human IgE, occupied by an oligomannose structure. This glycan is required for the conformational integrity of the IgE, and its removal ablates interactions with FcERI, thereby preventing allergic reactions and likely reducing IgE tissue half-life. This site on hIgE corresponds to the single glycosylation site found on IgG Fcs, which governs IgG Fc-mediated effector functions^{23,24}. Our findings demonstrate a similar functional requirement for glycosylation of IgE, supporting a close evolutionary relationship shared by these immunoglobulin classes²⁵. Because of its essential role in regulating IgE secondary structure, FcERI binding, mast cell activation, and consequently triggering of anaphylaxis, the oligomannose glycan on N394 in Cε3 of human IgE provides a novel therapeutic target for atopic and allergic diseases, as described herein.

### Materials and Methods

To induce polyclonal sera against OVA or peanut extracts, mice were injected with three doses of 10 ng antigen every seven days and sera were collected on day 10, 17, 19, and 21. Variable regions of OVA-specific antibodies were cloned from ToE hybridoma²⁶ and subcloned into constant regions of kappa light chain and IgE heavy chain. Recombinant aOVA-mIgE and aOVA-hIgE were generated by transient transfection using HEK293T, as described²⁷ and purified by OVA coupled to NHS agarose beads (GE HEALTHCARE). IgE was either genetically mutated at the N-linked glycan consensus sequences by site-directed mutagenesis (AGILENT) or treated with endoglycosidases PNGase F (NEW ENGLAND BIOLABS) or Endo F1 (SIGMA) to determine glycan contribution. mIgE or hIgE was quantitated by ELISA (BETHYL LABORATORIES). Site-specific glycosylation of recombinant aOVA-mIgE and aOVA-hIgE was identified by nano LC-MS/MS following enzymatic digestion of the proteins, as previously described²⁰. To test the ability of IgE to initiate PCA, IgE was injected intradermally into the ear followed by antigen challenge in PBS containing 2% Evans blue on the following day or 4 h later for mIgE or hIgE, respectively. Ear inflammation was determined by the level of blue dye in the ear extracted by dimethylformamide (EMD MILLIPORE). Flow cytometry was used to determine in vitro and in vivo binding of mIgE to the mast cells and hIgE binding to hFcεRI⁺ HeLa cells. ELISA was used to determine binding to antigen (OVA, DNP, and TNP) and saturation binding of hIgE to soluble hFcεRIα.

Mice. 5-6 weeks old BALB/c and C57BL/6 female mice were purchased from the Jackson Laboratory. hFcεRIα⁺/mFcεRIα^{-/-} and mFcεRIα^{-/-} mice¹ were maintained in the animal facility at MGH. Mice were all housed in specific pathogen-free conditions according to National Institutes of Health and all animal experiments were conducted under protocols approved by the Institutional Animal Care and Use Committee of Massachusetts General Hospital.

Passive cutaneous anaphylaxis. 20 ng of monoclonal mIgE specific for OVA, dinitrophenol (DNP; Clone SPE-7, SIGMA) or trinitrophenol (TNP; clone IgE-3, BD Pharmingen) or 5 ng of polyclonal IgE specific for OVA or peanut extracts were injected intradermally in the BALB/c mice ears and next day mice were intravenously challenged with PBS containing 125 μg of OVA (SIGMA), DNP-HSA (SIGMA), or TNP-BSA (conjugation ratio 13; BIOSEARCH TECHNOLOGIES), and 2% Evans blue dye in PBS. 45 min after challenge, the mice were sacrificed and the ears were excised and minced before incubation in N,N-dimethylformamide (EMD MILLIPORE) at 55° C. for 3 h. The degree of blue dye in the ears was quantitated by the absorbance at 650 nm. For studies of hIgE, 250 ng of αOVA-hIgE was injected intradermally in hFcεRIα⁺/mFcεRIα^{-/-} or mFcεRIα^{-/-} mice, followed by intravenous administration of OVA and Evan's Blue Dye 4 hours later.

IgE antibodies. The variable and constant regions of heavy and light chains were individually cloned from OVA-specific Toe hybridoma, adapting from the protocol previously described². Once the variable and the constant regions of heavy and light chain were joined by overlapping PCR, the heavy chain was placed under CMV promoter using restriction enzyme sites SalI and XbaI and the light chain was placed under EF1α promoter using restriction enzyme sites NotI and KpnI in pBUDCE4.1 expression vector (INVITROGEN). A similar cloning strategy was used to construct αOVA-hIgE vector. Constant regions of heavy and light chains of human IgE were cloned from αHEL-hIgE vector (from J. Ravetch). The N-linked glycosylation sequon of IgE were mutated using QUIKCHANGE II XL SITE-DIRECTED MUTAGENESIS KIT (AGILENT), following the manufacturer's protocol.

Recombinant antibodies were generated by transient transfection of the plasmids into HEK293T using Polyethylenimine “Max” (POLYSCIENCES, INC) followed by purification using N-hydroxysuccinimide-activated Sepharose beads (GE HEALTHCARE) coupled to OVA (SIGMA). Antibodies generated were verified by immunoblots for size, quantified by IgE ELISA and the specificity confirmed by OVA ELISA.

Polyclonal IgE specific for ovalbumin (OVA) or peanut extracts were prepared by injected BALB/c mice with 10 μg of OVA or peanut extract in aluminum hydroxide on days 0, 7, and 14. Mice were bled on days 10, 17, 19, and 21. The sera was separated from the blood by serum gel tubes (BD BIOSCIENCE), and depleted of IgG by incubating with Protein G high-capacity agarose (THERMO SCIENTIFIC).

Labelling and digestion of IgE. Human IgE (HE1 clone, ABCAM) was biotinylated using Biotin-XX Microscale Protein Labeling Kit (MOLECULAR PROBES). WT and N384Q αOVA-mIgE were conjugated to Alexa488 or Alexa567 by manufacturer's recommendations (MOLECULAR PROBES). OVA-A647 was from (MOLECULAR PROBES), and streptavidin-A647 from BIOLEGEND.

IgE were digested with PNGase F (NEW ENGLAND BIOLABS) or Endoglycosidase F1 (SIGMA) following manufacturer's instructions under non-denaturing conditions at 37° C. for 72 h. All digestions were verified by lectin blot.

Immuno- and lectin blotting. Immuno- and lectin blotting were performed as described previously³. In brief, equal amounts of protein were resolved on 3-8% Tris-Acetate Protein Gels (LIFE TECHNOLOGIES) in SDS-PAGE under non-reducing conditions. After transfer to polyvinylidene difluoride membranes, membrane were blocked with 5% dry milk in PBS containing 0.05% Tween 20 for immunoblotting or with Protein-Free Blocking Buffers (PIERCE) for lectin blotting. Mouse or human IgE were probed by goat polyclonal anti-mouse or anti-human IgE-HRP (10 ng/mL, BETHYL LABORATORIES), repsectively. N-linked glycans were detected by biotinylated Lens Culinaris Agglutinin (LCA, 5 μg/mL, VECTOR LABORATORIES) and α1,3 and α1,6 linked high mannose structures were detected by biotinylated Galanthus Nivalis lectin (GNA, 4 μg/mL, VECTOR LABORATORIES).

ELISA.

Mouse or human IgE were quantified by sandwich ELISA following instructions from Mouse or Human IgE ELISA Quantitation Set (BETHYL LABORATORIES). Antibodies specific for OVA was verified by 96-well Nunc plates plate coated with 75 μg/mL OVA (SIGMA), blocked with 2% BSA in PBS and probed with goat polyclonal anti-mouse or anti-human IgE-HRP (2 ng/mL, BETHYL LABORATORIES). A similar protocol was used for verifying antibodies specific for DNP and TNP, except 5 μg/mL of DNP-HSA (SIGMA) and TNP-BSA (conjugation ratio 13; BIOSEARCH TECHNOLOGIES) was used for coating. All reactions were detected by 3,3,5,5-tetramethylbenzidine (TMB, thereto scientific), stopped by 2 M sulphuric acid and the absorbance measured at 450 nm.

Human mast cell degranulation assay. Human LAD-2 mast cell line was cultured and degranulation measured as described previously^{4,5}. In brief, LAD-2 cells were sensitized with 250 ng of aOVA-hIgE or 100 ng of biotinylated hIgE overnight. Upon OVA or streptavidin (SIGMA) activation, the level of mast cells degranulation was monitored by the release of f3-hexosaminidase in mast cell granules, quantified by the extent of its substrate p-nitrophenyl N-acetyl-f3-D-glucosamide (PNAG) digested in a colorimetric assay.

Flow Cytometry IgE-FcεRI binding assay. Mouse bone marrow-derived mast cells (BMMCs) generated by flushing marrow from tibias and femurs, and culturing the cells in IL-3 for 3-5 weeks, as previously described. hFcdRIa+ HeLa cells have been previously described (REF). BMMCs or hFcdRIa+ HeLa cells were incubated overnight 100 ng of mouse or human IgE, respectively. The cells were washed the next day, and stained with Alexa647-OVA for OVA-specific IgE, Alexa647-streptavidin for biotinylated hIgE, or FcdRI by anti-hFcdRIa (CraI-PE) and anti-mFcdRIa (MarI-APC, EBIOSCIENCE), and analyzed by Flow Cytometry on a BD FACSCALIBUR. Histograms and MFI were generated and determined FLOWJO software (TREESTAR).

Saturation binding assay. The extracellular portion of the alpha chain of human FcERI (shFcERIa) was cloned from cDNA of human myeloid dendritic cells into HindIII and BamHI sites of p3x-FLAG-CMV-13 (SIGMA) to generate shFcERIa-flag. The plasmid was transiently transfected into HEK293T cells as described above, and shFcERIa-flag protein was purified from culture supernatants using anti-flag M2 affinity gel (SIGMA) per manufacturer's instructions. 96-well Nunc plates were coated with shFcERIa-flag (10 ng/pl), blocked with 2% BSA in PBS, and incubated with increasing concentrations of WT and N394Q aOVA-hIgE, or hIgE (ABCAM) and EndoF1 treated-hIgE. After 30 min, the wells were washed, and shFcERIa-flag-bound hIgE was probed by anti-Fab-HRP (BETHYL) and detected by TMB (THERMO SCIENTIFIC). The reactions were stopped by 2 M sulphuric acid and the absorbance measured at 450 nm.

Glycopeptide mass spectrometry analysis and data analysis. Site-specific glycosylation was quantified for both the recombinant aOVA-mIgE and aOVA-hIgE using nano LC-MS/MS following enzymatic digestion of the proteins, as previously described⁶. Most sites were quantified from the tryptic digest based on the extracted ion current for the most abundant charge state for each of the peptides, except chymotrypsin was used for the analysis of N140 and N168 from aOVA-hIgE and N166, N195 and N207 from aOVA-mIgE. The isolated IgE was prepared for proteolysis by denaturing the protein in 6M guanidine HCl followed by reduction with dithiothreitol and alkylation with iodoacetamide. The enzymatic digests were performed in 25 mM ammonium bicarbonate pH 7.8 overnight (trypsin) or for 4 h (chymotrypsin). The digestion was quenched with formic acid added to 2% w/w. The separation was performed on a Thermo EasySpray C18 nLC column 0.75 tm×50 cm using water and acetonitrile with 0.1% formic acid for mobile phase A and mobile phase B, respectively. A linear gradient from 1% to 35% mobile phase B was runs 120 min. Mass spectra were recorded on a Thermo QExactive mass spectrometer operated in positive mode and using a top 12 data dependent method. Glycopeptides were quantified based on the extracted ion area for abundant charge state for each glycopeptide. The relative abundance was calculated for all identified glycan species for each site using software.

Conformational analysis by circular dichroism. Untreated human IgE (HE1 clone, Abcam) or human IgE digested with Endo F1 or Endo F1 buffer only was exchanged into 10 mM sodium phosphate buffer, pH 7.0 and concentrated to 0.1 tg/tl. The CD spectra of IgE were acquired in Jasco J815 spectropolarimeter in a 1 mm quartz cell. Data was acquired in the far UV range (245-195 nm) in triplicates. At xx° C. with xx bandwidth and xx s. Ellipticity is expressed in molar circular dichroism (AE) where AE=O(Obs)×mean residue weight/10×solute concentration (C)×pathlength (1)×3298

Flow Cytometry.

Untreated ears or ears that were intradermally injected with fluorescently labeled aOVA-mIgE 16 h prior were separated into dorsal and ventral halves and minced before digestion with Liberase (ROCHE) and subjected to disruption, to generate single cell suspensions, as previously described⁷. Suspension cells were resuspended in PBS and incubated with Zombie Yellow Fixable Viability Kit (BIOLEGEND) before incubation with anti-mouse CD16/CD32 (clone 93) in FACS buffer (2 mM EDTA and 0.5% BSA in PBS). Antibodies for surface antigen staining include: Alexa647 anti-mouse CD117 (c-Kit; Clone 2B8; BIOLEGEND), Pacific Blue anti-mouse CD45 (Clone 30-F11; BIOLEGEND), PE/Cy7 anti-mouse/human CD11b (Clone M1/70; BIOLEGEND), FITC anti-mouse CD8 (Clone 53-6.7; BIOLEGEND), PE anti-mouse CD8 (Clone 53-6.7; BIOLEGEND), APC anti-mouse CD8 (Clone 53-6.7; BIOLEGEND), PE/Cy7 anti-mouse CD8 (Clone 53-6.7; BIOLEGEND). Cells were resuspended in FACS buffer after staining and acquired using LSRII flow cytometer (BD BIOSCIENCES). Data were analysed using FLOWJO Version 7.6 software (TREE STAR).

Statistical analyses. All statistical analyses were performed using PRISM 6 (GRAPHPAD) and results are shown as means with standard error of the mean (s.e.m.). An unpaired Student's t-test was used to compare two unmatched groups. For the comparison between three or more more groups, one-way or two-way ANOVA with Bonferroni's multiple comparisons test was used.

### REFERENCES

All references cited herein are incorporated by reference in their entirety.
1. World Allergy Organization (WAO) White Book on Allergy. 1-248 (World Allergy Organization, 2013).
2. Galli, S. J., Tsai, M. & Piliponsky, A. M. The development of allergic inflammation. Nature 454, 445-454 (2008).
3. Gould, H. J. & Sutton, B. J. IgE in allergy and asthma today. Nat Rev Immunol 8, 205-217 (2008).
4. Holgate, S. T. & Polosa, R. Treatment strategies for allergy and asthma. Nat Rev Immunol 8, 218-230 (2008).
5. Arnold, J. N., Wormald, M. R., Sim, R. B., Rudd, P. M. & Dwek, R. A. The impact of glycosylation on the biological function and structure of human immunoglobulins. Annu. Rev. Immunol. 25, 21-50 (2007).
6. Matsumoto, M. et al. IgG and IgE Collaboratively Accelerate Expulsion of Strongyloides venezuelensis in a Primary Infection. Infection and Immunity 81, 2518-2527 (2013).

Dessein, A. J., Parker, W. L., James, S. L. & David, J. R. IgE antibody and resistance to infection. I. Selective suppression of the IgE antibody response in rats diminishes the resistance and the eosinophil response to Trichinella spiralis infection. J Exp Med 153, 423-436 (1981).
8. Marichal, T. et al. A beneficial role for immunoglobulin E in host defense against honeybee venom. Immunity 39, 963-975 (2013).
9. Palm, N. W. et al. Bee venom phospholipase A2 induces a primary type 2 response that is dependent on the receptor ST2 and confers protective immunity. Immunity 39, 976-985 (2013).
10. Gould, H. J. et al. The biology of IgE and the basis of allergic disease. Annu. Rev. Immunol. 21, 579-628 (2003).
11. Galli, S. J. & Tsai, M. IgE and mast cells in allergic disease. Nat Med 18, 693-704 (2012).
12. Saini, S. S. et al. Efficacy and Safety of Omalizumab in Patients with Chronic Idiopathic/Spontaneous Urticaria Who Remain Symptomatic on H1-Antihistamines: A Randomized, Placebo-Controlled Study. J. Invest. Dermatol. (2014). doi:10.1038/jid.2014.306
13. Gauvreau, G. M. et al. Targeting membrane-expressed IgE B cell receptor with an antibody to the M1 prime epitope reduces IgE production. Sci Transl Med 6, 243ra85-243ra85 (2014).
14. Schwab, I. & Nimmerjahn, F. Intravenous immunoglobulin therapy: how does IgG modulate the immune system? Nat Rev Immunol 13, 176-189 (2013).
15. Dombrowicz, D. et al. Anaphylaxis mediated through a humanized high affinity IgE receptor. J Immunol 157, 1645-1651 (1996).
16. Morrison, S. L. Cloning, expression, and modification of antibody V regions. Curr Protoc Immunol Chapter 2, Unit 2.12-Un12 (2002).
17. Anthony, R. M. et al. Recapitulation of IVIG Anti-Inflammatory Activity with a Recombinant IgG Fc. Science 320, 373-(2008).
18. Kuehn, H. S., Radinger, M. & Gilfillan, A. M. Measuring mast cell mediator release. Curr Protoc Immunol Chapter 7, Unit7-Uni38 (2010).
19. Radinger, M. M., Jensen, B. M. B., Kuehn, H. S. H., Kirshenbaum, A. A. & Gilfillan, A. M.

A. Generation, isolation, and maintenance of human mast cells and mast cell lines derived from peripheral blood or cord blood. Curr Protoc Immunol Chapter 7, Unit-Un37 (2010).
20. Plomp, R. et al. Site-Specific N-Glycosylation Analysis of Human Immunoglobulin E. J. Proteome Res. 131206080654002 (2013). doi:10.1021/pr400714w
21. Riol-Blanco, L. et al. Nociceptive sensory neurons drive interleukin-23-mediated psoriasiform skin inflammation. Nature (2014). doi:10.1038/nature13199
22. Dombrowicz, D., Flamand, V., Brigman, K. K., Koller, B. H. & Kinet, J.-P. Abolition of anaphylaxis by targeted disruption of the high affinity immunoglobulin E receptor a chain gene. Cell 75, 969-976 (1993).
23. Kirshenbaum, A. S. et al. Characterization of novel stem cell factor responsive human mast cell lines LAD 1 and 2 established from a patient with mast cell sarcoma/leukemia; activation following aggregation of FIεRI or FcγRI. Leukemia Research 27, 677-682 (2003).
24. Dorrington, K. J. & Bennich, H. H. Structure-function relationships in human immunoglobulin E. Immunol. Rev. 41, 3-25 (1978).
25. Arnold, J. N. J. et al. The glycosylation of human serum IgD and IgE and the accessibility of identified oligomannose structures for interaction with mannan-binding lectin. J Immunol 173, 6831-6840 (2004).
26. Plomp, R. et al. Site-Specific N-Glycosylation Analysis of Human Immunoglobulin E. J. Proteome Res. 131206080654002 (2013). doi:10.1021/pr400714w
27. Feige, M. J. et al. Structure of the Murine Unglycosylated IgG1 Fc Fragment. J Mol Biol 391, 599-608 (2009).
28. Sondermann, P., Pincetic, A., Maamary, J., Lammens, K. & Ravetch, J. V. General mechanism for modulating immunoglobulin effector function. Proc Natl Acad Sci USA 110, 9868-9872 (2013).
29. Garman, S. C., Wurzburg, B. A., Tarchevskaya, S. S., Kinet, J. P. & Jardetzky, T. S. Structure of the Fc fragment of human IgE bound to its high-affinity receptor Fc epsilonRI alpha. Nature 406, 259-266 (2000).
30. Pincetic, A. et al. Type I and type II Fc receptors regulate innate and adaptive immunity. Nat Immunol 15, 707-716 (2014).
31. Flajnik, M. F. Comparative analyses of immunoglobulin genes: surprises and portents. Nat Rev Immunol 2, 688-698 (2002).
32. SAWADA, K. et al. The expression of murine eutaneous late phase reaction requires both IgE antibodies and CD4 T eells. Clin Exp Allergy 27, 225-231 (1997)
33. Nimmerjahn, F. & Ravetch, J. V. Divergent immunoglobulin g subclass activity through selective Fc receptor binding. Science 310, 1510-1512 (2005).

1. A method of inhibiting or blocking functional activity of IgE comprising administering to a subject in need thereof an inhibitor of glycosylation or an IgE-specific glycosylation inhibitor.

2. A method of treating an allergic or atopic disease in a subject in need thereof comprising administering to a subject in need thereof an inhibitor of glycosylation or an IgE-specific glycosylation inhibitor, wherein said inhibitor blocks functional activity of IgE, thereby treating an allergic or atopic disease.

3. The method of claim 1, wherein the IgE-specific glycosylation inhibitor blocks or inhibits glycosylation at the glycosylation sequon at N394 of IgE.

4. The method of claim 3, wherein the IgE-specific glycosylation inhibitor blocks or inhibits the glycosylation sequon at N394 of IgE and prevents or inhibits IgE-mediated signaling.

5. The method of claim 3, wherein the IgE-specific inhibitor is an antibody or antigen-binding fragment thereof that specifically binds or is directed against the glycosylation sequon at N394 of IgE.

6. The method of claim 3, wherein the IgE-specific inhibitor is an antibody or antigen-binding fragment thereof that specifically binds or is directed against a site on a target in the proximity of the glycosylation sequon at N394 of IgE, thereby providing steric hindrance for the interaction of IgE with its receptor.

7. The method of claim 3, wherein the antibody or antigen-binding fragment thereof that specifically binds or is directed against the glycosylation sequon at N394 of IgE, does not bind to IgE when bound to either of the Fcε receptors.

8. The method of claim 1, wherein the IgE-specific inhibitor is a small molecule compound that binds to the glycosylation sequon at N394 of IgE, thereby inhibiting or preventing IgE binding to either of the FCε receptors.

9. The method of claim 8, wherein the small molecule inhibitor specifically binds the glycosylation sequon at N394 of IgE when glycosylated or specifically binds the glycosylation sequon at N394 of IgE when unglycosylated, and prevents the glycosylation at N394.

10. The method of claim 1, wherein the glycosylation inhibitor is selected from tunicamycin, a tunicaymycin homolog, streptovirudin, mycospocidin, amphomycin, tsushimycin, antibiotic 24010, antibiotic MM 19290, bacitracin, corynetoxin, showdomycin, duimycin, 1-deoxymannonojirimycin, deoxynojirimycin, N-methyl-1-dexoymannojirimycin, brefeldin A, a glucose analog, a mannose analog, 2-deoxy-D-glucose, 2-deoxyglucose, D-(+)-mannose, D-(+) galactose, 2-deoxy-2-fluoro-D-glucose, 1,4-dideoxy-1,4-imino-D-mannitol (DIM), fluoroglucose, fluoromannose, UDP-2-deoxyglucose, GDP-2-deoxyglucose, a hydroxymethylglutaryl-CoA reductase inhibitor, 25-hydroxycholesterol, hydroxycholesterol, swainsonine, cycloheximide, puromycin, actinomycin D, monensin, m-Chlorocarbonyl-cyanide phenylhydrazone (CCCP), compactin, dolichyl-phosphoryl-2-deoxyglucose, N-Acetyl-D-Glucosamine, hypoxanthine, thymidine, cholesterol, glucosamine, mannosamine, castanospermine, glutamine, bromoconduritol, conduritol epoxide, a conduritol derivative, aglycosylmethyl-p-nitrophenyltriazene, β-Hydroxynorvaline, threo-β-fluoroasparagine, D-(+)-Gluconic acid.delta.-lactone, di(2-ethyl hexyl)phosphate, tributyl phosphate, dodecyl phosphate, 2-dimethylamino ethyl ester of (diphenyl methyl)-phosphoric acid, [2-(diphenyl phosphinyloxy)ethyl]trimethyl ammonium iodide, iodoacetate, fluoroacetate, Kinefusine, Australine, Castanospermine, Deoxynojirimycin, Deoxymannojirimycin, Swainsoninne, and Mannostatin A.

11. The method of claim 1, wherein the glycosylation inhibitor is selected from an inhibitor of N-linked glycan biosynthesis, an inhibitor of N-linked glycan N-acetylglucosaminyl transferase, an inhibitor of N-linked glycan fucosyl transferase, an inhibitor of N-linked glycan galactosyl transferase, an inhibitor of N-linked glycan sialyl transferase, an inhibitor of N-linked glycan sulfotransferase, an inhibitor of N-linked glycanglycophosphotransferase, or a combination thereof.

12. The method of claim 1, wherein the glycosylation inhibitor is not EndoS.

13. The method of claim 1, wherein the allergic or atopic disorder is selected from allergic asthma, eczema, allergic rhinitis or coryza, hay fever, bronchial asthma, urticaria (hives), gastrointestinal allergies, eosinophilia, conjunctivitis, and glomerulonephritis.


SEQUENCE LISTING (16 sequences)

SEQ ID NO: 1 - protein, 3 aa, Artificial Sequence
  Description of Artificial Sequence: Synthetic peptide
  MOD_RES (2)..(2): Any amino acid except proline
NXS

SEQ ID NO: 2 - protein, 3 aa, Artificial Sequence
  Description of Artificial Sequence: Synthetic peptide
  MOD_RES (2)..(2): Any amino acid except proline
NXT

SEQ ID NO: 3 - protein, 3 aa, Artificial Sequence
  Description of Artificial Sequence: Synthetic peptide
  MOD_RES (2)..(2): Any amino acid except proline
NXC

SEQ ID NO: 4-15 - skipped (12 entries)

SEQ ID NO: 16 - protein, 3 aa, Artificial Sequence
  Description of Artificial Sequence: Synthetic peptide
  MOD_RES (2)..(2): Any amino acid
  MOD_RES (3)..(3): Ser or Thr
NXX